(19) United States
(12) Patent Application Publication
(10) Pub. No.: US 2011/0010100 A1
(43) Pub. Date: Jan. 13, 2011

(54) FUNCTIONAL DOMAIN ANALYSIS METHOD AND SYSTEM

(76) Inventors: Yixue Li, Shanghai (CN); Pei Hao, Shanghai (CN); Yun Li, Tongxiang (CN)

Correspondence Address: PERKINS COIE LLP, POST OFFICE BOX 1208, SEATTLE, WA, 98111-1208, US

(21) Appl. No.: 12/498,553

(22) Filed: Jul. 7, 2009

(51) Int. Cl. G01N 33/48; G06F 19/00; G06F 17/18

(52) U.S. Cl. 702/19; 702/181

(57) ABSTRACT

One embodiment of the present disclosure relates to methods for assessing the biological effects of a test agent or test condition on a test sample by comparing one or more characteristics of the biomarkers of the test sample with that of one or more reference samples and assessing the biological effects of the test agent or test condition based on the biological effects of one or more reference agents or reference conditions on the one or more reference samples, as well as computer program products for executing such methods, computer readable storage media encoding such computer programs, and computer systems for performing such methods.

### BACKGROUND

Biological samples are sometimes measured by their biomarkers to examine their biological functions. Effects of a therapeutic compound on a subject or a biological sample may also be detected through measuring biomarkers of the subject or biological sample.

### SUMMARY

In one aspect, the present disclosure provides a method for assessing the biological effects of a test agent or a test condition, which comprises identifying one or more target biomarkers of a test sample contacted with the test agent or in the test condition, grouping the one or more target biomarkers into one or more target functional domains according to pre-determined criteria, identifying one or more reference samples having relevance to the test sample, and assessing the biological effects of the test agent or test condition based on biological effects of one or more reference agents or reference conditions on the one or more reference samples.

In another aspect, the present disclosure provides a computer program product comprising one or more instructions recorded on a machine-readable recording medium for assessing the biological effects of a test agent or a test condition as described in the present disclosure.

In another aspect, the present disclosure provides a computer readable storage medium having a computer program encoded thereon, wherein said computer program when executed by a computer instructs the computer to execute a method for assessing the biological effects of a test agent or a test condition as described in the present disclosure.

In another aspect, the present disclosure provides a system for assessing the biological effects of a test agent or a test condition, comprising one or more input devices, one or more output devices, one or more processors, and one or more memory devices storing therein one or more operating systems, one or more computer programs, and one or more databases, interconnected by a bus.

The foregoing summary is illustrative only and is not intended to be in any way limiting. In addition to the illustrative aspects, embodiments and features described above, further aspects, embodiments and features will become apparent by reference to the figures and the following detailed description. Further, all U.S. patents or other references cited below are incorporated herein in their entirety by reference.

### BRIEF DESCRIPTION OF THE DRAWINGS

FIG. 1 is a flow chart showing an illustrative embodiment of a method for assessing the biological effects of a test agent or a test condition described in this disclosure.

FIG. 2 shows a flow chart of an illustrative embodiment of identifying, one or more target biomarkers of a test sample contacted with the test agent or in the test condition.

FIG. 3 shows a flow chart of an illustrative embodiment of identifying one or more target genes of a test sample contacted with the test agent or in the test condition.

FIG. 4 shows a flow chart of an illustrative embodiment of identifying one or more enriched target functional domains.

FIG. 5 shows a flow chart of an illustrative embodiment of identifying one or more reference samples.

FIG. 6 shows an illustrative computer interface for rankings of reference samples obtained using a method described herein.

FIG. 7 shows an illustrative computer output display obtained using a method described herein. Empty circles represent functional domains in which the test sample shows positive relevance to the reference sample, filled circles represent functional domains in which the test sample shows negative relevance to the reference sample, and dotted circles represent functional domains in which the test sample shows no relevance to the reference sample.

FIG. 8 shows an illustrative computer output display obtained using a method described herein. The results are shown in a table, wherein the columns correspond to enriched target functional domains of the test sample, and the rows correspond to the reference samples. The relevance of each reference sample to each enriched target functional domain is shown in the cells of the table. An empty cell, filled cell, and dotted cell indicate that the reference sample and the enriched target functional domain have positive relevance, negative relevance, and no relevance, respectively.

FIG. 9 shows a schematic diagram of an illustrative embodiment of hard ware structures of a computer system described in this disclosure.

FIG. 10 shows an illustrative computer interface for log₂ transformed raw data obtained from a microarray analysis.

### DETAILED DESCRIPTION

In the following, detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the Figures, can be arranged, substituted, combined, and designed in a wide variety of different configurations, all of which are explicitly contemplated and make part of this disclosure.

Exposure of biological samples to one or more agents may cause changes in the biological functions of the sample that may be measured and/or identified at least partially based on biomarkers associated with the sample. Similarly, biological samples isolated and/or extracted from cells/tissues/subjects exposed to a condition may exhibit changes in the biological functions of the sample that may be measured and/or identified at least partially based on biomarkers associated with the sample. To assess the effects of a test agent or test condition on the biological functions of a test sample, one or more characteristics of biomarkers of the test sample may be measured and/or identified, and compared with one or more characteristics of biomarkers in a reference sample.

Relevance between the one or more characteristics of the biomarkers of the test sample and the one or more characteristics of the biomarkers of the reference sample may be determined. The relevance indicates whether the one or more biological effects of the test agent/condition on the test sample can be related to the one or more biological effects of the reference agent/condition on the reference sample. In the reference samples, the biological effects of the reference agent/condition on the reference sample are already known and/or are separately obtained. Therefore, the biological effects of the test agent/condition may be assessed based on information regarding the biological effects of the reference agent/condition on the reference sample.

The present disclosure provides, among others, methods, computer programs, computers, and systems, for assessing the biological effects of a test agent/condition on a test sample by comparing one or more characteristics of one or more biomarkers of the test sample contacted with the test agent or in the test condition with one or more characteristics of one or more biomarkers of one or more reference samples contacted with one or more reference agents or in one or more reference conditions.

In certain embodiments, the present disclosure provides a method for assessing the biological effects of a test agent/condition on a test sample, the method comprising identifying one or more target biomarkers of a test sample contacted with the test agent or in the test condition, grouping the one or more target biomarkers into one or more target functional domains according to predetermined criteria, identifying one or more reference samples having relevance to the test sample, and assessing the biological effects of the test agent/condition based on the biological effects of one or more reference agents/conditions on the one or more reference samples.

The test sample may include any biological materials, including, without limitation, molecules, cells and tissues from human beings, animals, plants, and microorganisms. In illustrative embodiments, molecules may include, but are not limited to, proteins, peptides, nucleic acids, nucleotides, lipids, compounds, metabolites, carbohydrates, saccharides, lipoproteins, glycoproteins, biological complexes such as protein-DNA complexes (e.g. chromosomes), protein-lipid complexes, and protein-protein complexes. In illustrative embodiments, cells may include, but are not limited to, cultured cells, cells obtained through biopsy, skin scraping, and/or other medical or surgical procedures, tumor cell lines such as acute myeoblastic leukemia cells OCI/AML2, MCF-7 cells, Hela cells, hybridoma cell lines, stem cells. Jurkat cells, B cells, glial cells, hepatocytes, myocardiocytes, spleen cells, CHO cells, and 293 cells. Illustrative embodiments of tissues include, but are not limited to, skin tissues, liver tissues, kidney tissues, muscle tissues, bone tissues, lung tissues, brain tissues, blood tissues, bone marrow tissues, and other types of tissue samples from human beings, animals (including animal disease models such as animals implanted with tumor tissues), and plants.

The test agent may be any physical substance, including, without limitation, one or more chemical compounds, one or more biological agents such as recombinant nucleic acids and proteins, one or more herbal medicines, one or more nutritional supplements, and one or more food products. Illustrative examples include drugs such as anti-cancer drugs, anti-inflammatory drugs, antibiotics or such drug candidates, insulin or its derivative peptides, human growth hormone or its derivative peptides, anti-sense RNA, siRNA, antibodies, vaccines, vitamins, etc.

The test condition may be any biological or physiological state, including, without limitation, a disease, disorder, physical stimulation, physical condition such as temperature and pressure. Illustrative examples include cancers, heart diseases, flu, high blood pressure, stress, anxiety, hypothermia. etc.

The test agent/condition may exert various biological effects on the test sample, including, without limitation, molecular, cellular and/or any other biological effects, which may be measured and/or identified at least partially based on the biomarkers of the test sample. Illustrative examples of biological effects include stimulation or inhibition of cell growth, stimulation or inhibition of cell signaling pathways such as the MAPK pathway and the JNK pathway, activation or inhibition of transcription factors such as nuclear factor kappa-light-chain-enhancer of activated B cells (NF-kB) and Signal Transducers and Activators of Transcription (STAT) proteins, etc.

A reference sample is a biological sample contacted with a reference agent or in a reference condition in which the biological effects of the reference agent/condition on the reference sample are assessed or evaluated or otherwise known. The reference sample may include any biological materials, including, without limitation, those described above, such as molecules, cells and tissues from human beings, animals, plants, and microorganisms. The reference agent may be any physical substance such as those described above, including, without limitation, one or more chemical compounds, one or more biological agents such as recombinant nucleic acids and proteins, one or more herbal medicines, one or more nutritional supplements, and one or more food products. The reference condition may be any biological or physiological state such as those described above, including, without limitation, a disease, disorder, physical stimulation, physical condition such as temperature and pressure. The reference agent/condition may exert various biological effects on the reference sample, including, without limitation, molecular, cellular and/or any other biological effects, which may be monitored by measuring the biomarkers of the reference sample.

The reference sample and the test sample shall be sufficiently comparable to each other to allow a person with ordinary skill in the art to compare the characteristics of the biomarkers of the reference sample and the characteristics of the biomarkers of the test sample and to assess and evaluate the biological effects of test agents/conditions on the test sample based on the biological effects of reference agents/conditions on the reference sample. For example, without limitation, the reference sample may contain the same type of biological materials as the test sample, and/or the same type of biomarkers and biomarker characteristics are measured and/or identified in the reference sample and the test sample. In illustrative embodiments, the reference sample may be one or more wild type and/or normal cells and optionally known cancerous cells of one type, while the test sample may be the same type of cell that is suspected of being cancerous. In illustrative embodiments, the reference sample may be one or more wild type and/or normal cells, and optionally cells of the same type under a series of physiological stresses, while the test sample may be the same type of cell under some form of physiological stress, for example. In illustrative embodiments, the same type of cell may include for example liver cells (or cells of any tissue—skin, blood, bone marrow, etc) isolated from one patient, or from different patients; or in some cases from the same species or from different species.

The term “biomarker” refers to any molecular or cellular substance of a biological material that may be used to indicate or measure biological features or functions of such biological material. A biomarker may include, without limitation, a gene (DNA or RNA), protein, carbohydrate structure, and glycolipid. In an illustrative embodiment, chromosomes and DNA sequences are used to identify genetic diseases such as Downs Syndrome and Haemophilia. In another illustrative embodiment, mRNA is used to measure protein expression levels and/or the presence or absence of proteins such as cell signaling factors (e.g. vascular endothelial growth factor (VEGF) and epidermal growth factor (EGF)) and nuclear transcription factors (e.g. NF-kB and STAT proteins). In yet another illustrative embodiment, antibodies are used for the detection of exposure to pathogens such as human immunodeficiency virus (HIV), hepatitis B virus (HBV) and syphilis. In yet another illustrative embodiment, blood glucose levels may be used for measuring the effects of insulin, etc. “Test biomarkers” refers to biomarkers of test samples, and “reference biomarkers” refers to biomarkers of reference samples.

A biomarker may have one or more characteristics that can be measured and/or identified to demonstrate the biological effects of a test agent/condition on a test sample or the biological effects of a reference agent/condition on a reference sample. The characteristics of a biomarker may include, but are not limited to, the amount of the biomarker present in a sample, the presence or absence of a biomarker in a sample, the activation state of the biomarker in a sample (e.g. phosphorylation of the biomarker, glycosylation of the biomarker), change in the amino acid or nucleic acid sequences of the biomarker (e.g. change from premature protein to mature protein, gene mutations, protein variants). In an illustrative embodiment, the amounts of mRNA in a cell are measured as an indication of protein expression levels in the cells. In another illustrative embodiment, blood glucose concentrations may be measured as an indication of the body's ability to regulate blood sugar levels. In yet another illustrative embodiment, the phosphorylation status of various kinases in a cell is measured to monitor the biological functions of the cell.

The characteristics of a biomarker may be measured and/or identified by methods known in the art. In an illustrative embodiment, the amount of mRNA expressed in a cell is measured using commercially available DNA chips (e.g. GeneChips of Affymetrix, Santa Clara, Calif.). In another illustrative embodiment, the amount of protein expressed in a cell is measured using commercially available protein chips (e.g. Ab Microarray 380 of Clontech Laboratories, Inc., Mountain View, Calif.). In another illustrative embodiment, the amount of glucose in a blood sample is measured using commercially available glucose assay products (e.g. Glucose Assay Kit of Cayman Chemical, Ann Arbor, Mich.). In another illustrative embodiment, the phosphorylation state of proteins is measured using known methods (e.g. Phospho-Bcl-2[pSer70] ELISA of Sigma-RBI, St. Louis, Mo.).

Relevance between the one or more characteristics of the biomarkers of the test sample and one or more characteristics of the biomarkers of the reference sample indicates the existence of a biological correlation (either positively correlated or negatively correlated) between the one or more characteristics of the biomarkers of the test sample and one or more characteristics of one or more biomarkers of the reference sample. Positive correlation suggests that the test agent/condition has biological effects on the test sample that are similar to the biological effects that the reference agent/condition has on the reference sample. Negative correlation suggests that the test agent/condition has biological effects on the test sample that are different from or opposite to the biological effects that the reference agent/condition has on the reference sample. For illustration, the correlation or lack of correlation may provide information regarding the presence or absence of an underlying disease in the test sample, provide information regarding possible toxicity of a test substance, or provide information regarding the potential activity and/or mechanism of action of a particular test substance.

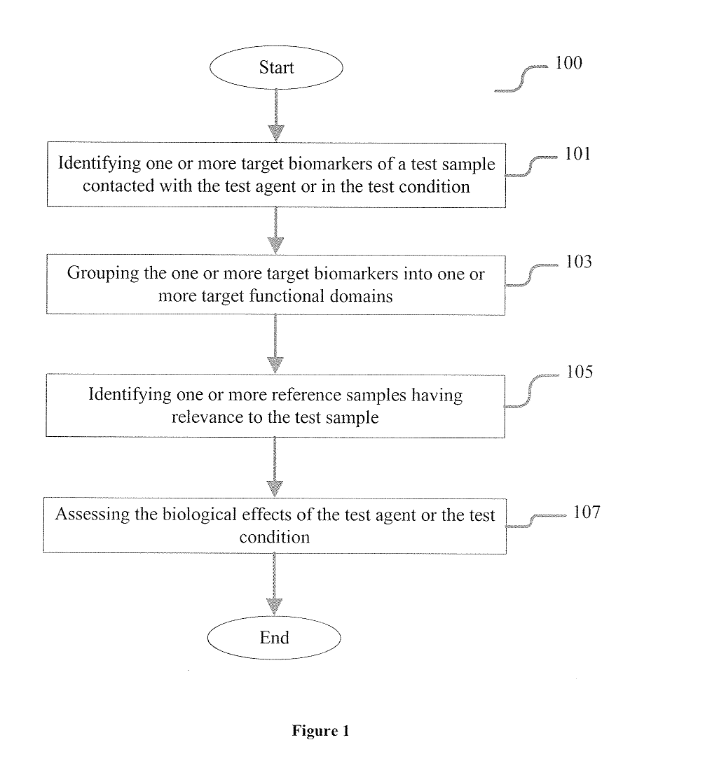

FIG. 1 shows an operational flow 100 representing an illustrative embodiment of operations of a method for assessing the biological effects of a test agent/condition provided in the present disclosure. As shown in FIG. 1, the method includes a target identification operation 101, that includes identifying one or more target biomarkers of a test sample; a grouping operation 103, that includes grouping the one or more target biomarkers into one or more target functional domains; a reference identification operation 105, that includes identifying one or more reference samples; and an assessing, operation 107, that includes assessing the biological effects of the test agent/condition.

In FIG. 1 and in the following figures that include various illustrative embodiments of operational flows, discussion and explanation may be provided with respect to methods and apparatus described herein, and/or with respect to other examples and contexts. The operational flows may also be executed in a variety of other contexts and environments, and/or in modified versions of those described herein. In addition, although some of the operational flows are presented in sequence, the various operations may be performed in various repetitions, concurrently, and/or in other orders than those that are illustrated.

In the target identification operation 101, one or more target biomarkers are selected from the one or more biomarkers of the test sample. The term “target biomarker” refers to a biomarker of the test sample that shows changes in one or more characteristics of the biomarker after the test sample is contacted with the test agent or is exposed to the test condition. The test sample may contain a plurality of biomarkers that show changes in one or more of their characteristics after being contacted with the test agent or being in the test condition. The biomarkers of the test sample that show changes in one or more of their characteristics may constitute a set of target biomarkers of the test sample.

The changes in one or more characteristics of the biomarkers of the test sample can be determined by analyzing the one or more characteristics of the biomarkers of the test samples with and without the test sample being contacted with the test agent or getting into the test condition. Data representing the one or more characteristics of the biomarkers of the test sample without contacting with the test agent or without onset of the test condition is typically control data. Data representing the one or more characteristics of the biomarkers of the test sample contacted with the test agent or with onset of the test condition is typically test data.

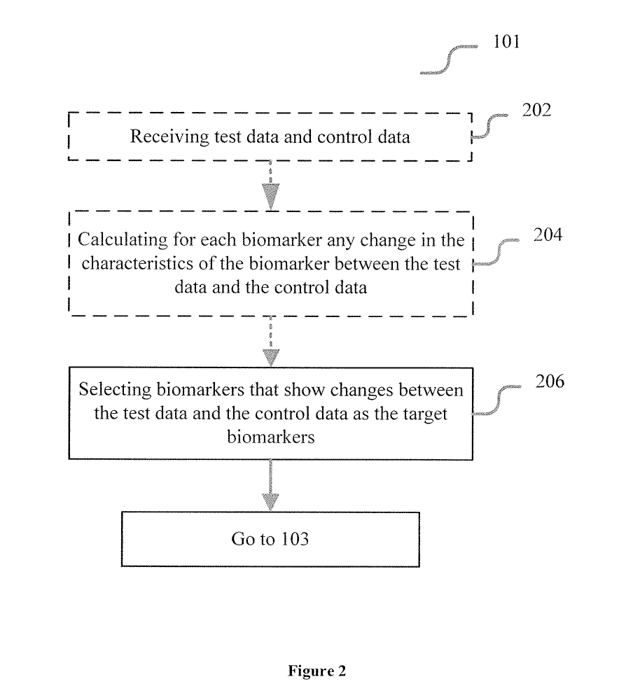

FIG. 2 shows an illustrative embodiment of the target identification operation 101, including an optional operation 202, that includes receiving test data and control data; an optional operation 204, that includes calculating for each biomarker any change in the characteristics of the biomarker between the test data and the control data; and an operation 206, that includes selecting biomarkers that show changes between the test data and the control data as the target biomarkers. In the optional operation 202 a set of test data and a set of control data are received. The test data and the control data may optionally be filtered, smoothed or undergo other data pre-processing treatments known in the art to remove or reduce background noises (see for example, Schuchhardt, J. et al. Normalization strategies for cDNA microarrays, Nucleic Acids Research, 28 (10):E47 (2000); Troyanskaya, O et al., Missing value estimation methods for DNA microarrays, Bioinformatics, 17: 520-525 (2001)).

Flowing from optional operation 202, in the optional operation 204, the test data is compared with the control data to determine the changes in the one or more characteristics of the biomarkers after the test sample is contacted with the test agent or is put in the test condition. The numerical values of the test data of the biomarkers may be larger than (or increased from) the numerical values of the control data of such biomarker, indicating that the changes in the one or more characteristics of the biomarkers are positive. The numerical values of the test data of the biomarkers may be smaller than (or decreased from) the numerical values of the control data, indicating that the changes in the one or more characteristics of the biomarkers are negative. Changes in the one or more characteristics of the biomarkers may be calculated by any method known in the art.

In an illustrative embodiment, the changes are calculated for each biomarker as the difference between the test data of the biomarker and the control data of such biomarker. In another illustrative embodiment, the changes are calculated for each biomarker as the percentage of the difference between the test data and the control data over the control data. In another illustrative embodiment, the changes are calculated for each biomarker as the ratio of the test data of the biomarker to the control data of such biomarker. In another illustrative embodiment, the changes are calculated for each biomarker as the logarithms of the ratio of the test data of the biomarker to the control data of such biomarker, in which, the base of the logarithm may be 2, 10, e or any other suitable number. In another illustrative embodiment, the changes are calculated as the differences or percentages of differences or ratios or logarithms of ratios of the ratios within the biomarkers of the test sample and the ratios within the biomarkers of the control sample.

One or more characteristics of a biomarker may occur or disappear in the test sample after the test sample is contacted with the test agent or is put in the test condition. Such presence or absence of one or more characteristics of a biomarker may also be shown by changes in the characteristics of the biomarker, for example, without limitation, by the difference between the test data and the control data. It would be obvious to a person with ordinary skill in the art that any other suitable calculation method for indicating the presence or absence of the biomarkers may be used.

Flowing from optional operation 204, the method processing goes to operation 206. In operation 206, biomarkers of the test sample that show changes in one or more of their characteristics after the test sample is contacted with the test agent or get into the test condition are selected as target biomarkers. In certain embodiments, target biomarkers may show substantial changes in their characteristics after the test sample is contacted with the test agent or get into the test condition. “Substantial change” means that the change in the characteristics of a biomarker in the test sample in comparison with the control sample is no less than a pre-selected threshold if the change is positive or no more than a pre-selected threshold if the change is negative.

The pre-selected threshold may be any suitable value that a person with ordinary skill in the art may determine as a reasonable threshold for showing the above background level changes in the characteristics of the biomarkers of the test sample in comparison with the control sample. Background level changes refer to changes that are caused by factors other than the test agent or test condition, such as for example, experimental errors, equipment errors, and inherent variation among different test samples. In an illustrative embodiment, if the changes in the characteristics of the biomarkers are calculated as the difference between the test data and the control data, then the pre-selected threshold may be 0.05, 0.1, 0.5, 1, 2, 5, 10, 20, 50 or 100 when the changes are positive and −0.05, −0.1, −0.5, −1, −2, −5, −10, −20, −50 or −100 when the changes are negative. In another illustrative embodiment, if the changes in the characteristics of the biomarkers are calculated as the percentages of the differences of the test data and the control data over the control data, then the pre-selected threshold may be 10%, 20%, 50%, 100% or 200% of the control data when the test data show increases from the control data, and may be −10%, −20%, −50%, −100% or −200% of the control data when the test data show decreases from the control data. In another illustrative embodiment, if the changes in the characteristics of the biomarkers are calculated as the ratios of the test data to the control data, then the pre-selected threshold may be 1.5, 2, 3, 5 or 10 when the test data show increases from the control data, and may be 2/3, 1/2, 1/3, 1/5 or 1/10 when the test data show decreases from the control data. In another illustrative embodiment, if the changes in the characteristics of the biomarkers are reflected by log₂ R wherein R is the ratio of the test data of a biomarker to the control data of such biomarker, then the pre-selected threshold may be 0.5, 1, 1.5, 2, 2.5 or 3 when the test data show increases from the control data, or −0.5, −1, −1.5, −2, −2.5 or −3 when the test data show decreases from the control data.

In certain embodiments, identifying one or more target biomarkers of a test sample comprises: receiving a set of test data representing one or more characteristics of one or more biomarkers of the test sample contacted with the test agent or in the test condition and a set of control data representing the characteristics of one or more biomarkers of the test sample not contacted with the test agent or in the test condition; calculating changes between the test data and the control data for the one or more biomarkers of the test sample; and selecting one or more target biomarkers of the test sample, wherein each target biomarker shows changes in the biomarker characteristics between the test data and the control data.

Flowing from the target identification operation 101, the method processing goes to the grouping operation 103, as shown in FIG. 1. In the grouping operation 103, the target biomarkers are grouped into one or more target functional domains according to pre-determined criteria. The term “target functional domain” refers to a group of one or more target biomarkers that share similar biological features and/or functions. The “pre-determined criteria” are criteria for dividing the target biomarkers into one or more target functional domains based on the biological features and/or functions of the target biomarkers. In an illustrative embodiment, one or more target biomarkers that are genes involved in cell cycle regulation are grouped together into a target functional domain Mᵢ; one or more target biomarkers that are genes involved in development are grouped together into a target functional domain Mⱼ; one or more target biomarkers that are genes involved in signal transduction are grouped together into a target functional domain Mₖ. A target biomarker may have one or more characteristics that fall into more than one target functional domain. In that case, a target biomarker may be grouped into more than one target functional domain. In an illustrative embodiment, one or more target biomarkers that are genes involved in both cell cycle regulation and signal transduction are grouped into a target functional domain Mᵢ for cell cycle regulation and a target functional domain Mₖ for signal transduction. The target biomarkers of a test sample may be grouped into target functional domains according to any suitable biological classification criteria. The classification criteria may be based on molecular or cellular functions, metabolic pathways, biological processes, cellular localizations, physiological functions, or any other biologically or physiologically meaningful classification of the biomarkers. In an illustrative embodiment, gene ontology and protein ontology, which are existing classification methods based on biological features and functions, are used to classify biomarkers into target functional domains.

Gene ontology, as illustrated in http://www.geneontology.org, provides structured, controlled vocabularies and classifications that cover several domains of molecular and cellular biology and are available for use in the annotation of genes, gene products and sequences. It uses ontologies to describe attributes of gene products in three non-overlapping domains of molecular biology: molecular function, biological process, and cellular component (see The Gene Ontology (GO) database and informatics resource, Gene Ontology Consortium, Nucleic Acids Res., 32: D258-D261 (2004); see also The Gene Ontology Consortium, Gene Ontology: tool for the unification of biology. Nat Genet., 25: 25-29. (2000); The Gene Ontology Consortium, Creating the gene ontology resource: design and implementation, Genome Res., 11: 1425-1433 (2001); Blake et al., The Gene Ontology (GO) project: structured vocabularies for molecular biology and their application to genome and expression analysis. Curr Protoc Bioinformatics, Chapter 7: Unit 7.2. (2003)). Gene ontology can be used to group, for example, micro-array data, according to the biological functions and characteristics of the genes (see Li et al, Microarray Data Mining Using Gene Ontology, Stud Health Technol Inform.;107(Pt 2):778-82. (2004)). Illustrative functional domains classified by gene ontology include genes functioning in cell cycle, genes functioning in developmental process, genes functioning in signal transduction, genes functioning in cell communication, genes functioning in chemotaxis, genes functioning in reproduction, genes functioning in immune response, genes functioning in adaptive immune response, genes functioning in response to stress, genes functioning in response to wounding, genes functioning in behavior, etc.

Protein ontology, as illustrated in Structural Classification of Proteins (SCOP) database (http://scop.mrc-lmb.cam.ac.uk/scop/index.html), classifies proteins according to certain traits such as protein structures (see also Murzin et al., SCOP: a Structural Classification of Proteins database for the investigation of sequences and structures. J. Mol. Biol., 247, 536-540. (1995); Andreeva et al., SCOP database in 2004: refinements integrate structure and sequence family data, Nucleic Acids Res. 32:D226-9. (2004)). Illustrative functional domains classified by protein ontology include proteins with alpha helices, proteins with beta sheets, proteins with spectrin repeat-like motif proteins with long-alpha hairpin motif proteins with LEM/SAP HeH motif proteins with DNA/RNA-binding 3-helical bundle, proteins with ISP domain, proteins with SH3-like barrel, proteins with UBA domain, proteins with nucleotide-binding domain, etc.

Kyoto Encyclopedia of Genes and Genomes (KEGG) is a metabolic pathway database as previously illustrated (Goto, S et al, Organizing and computing metabolic pathway data in terms of binary relations, Pacific Symp. Biocomputing, 1997, 175-186). KEGG is available at http://www.genome.jp/kegg/, and can be used to group molecules into functional domains based on the metabolic pathways they are involved in. The molecules to be grouped may include genes, gene products, metabolic compounds, and any other molecules in a cell. Illustrative functional domains classified by KEGG include, molecules involved in carbohydrate metabolism pathway, molecules involved in citrate cycle, molecules involved in pentose phosphate pathway, molecules involved in energy metabolism, molecules involved in photosynthesis, molecules involved in lipid metabolism, molecules involved in fatty acid biosynthesis, molecules involved in nucleotide metabolism, and molecules involved in purine metabolism, etc.

Pathway interaction database (PID) can be used to group molecules into functional domains according to the signal pathways they participate in. The database is described in a research paper (Schaefer, Carl et al, PID: the pathway interaction database, Nucleic acids research, 2009, 37, D674-D679) and is available at http://pid.nci.nih.gov. The molecules which can be grouped using PID may include, small molecule compounds, RNAs, proteins and complex. Illustrative functional domains classified by PID include molecules participated in BCR signal pathway, molecules participated in Arf6 signaling events, molecules participated in Arf6 trafficking events, molecules participated in class I PI3K signaling events, molecules participated in PI3K non-lipid kinase events, molecules participated in EPO signaling pathway, molecules participated in IL-1 mediated signaling events, and molecules participated in caspase cascade in apoptosis, etc.

In an illustrative embodiment, the one or more target biomarkers in a target functional domain are further analyzed to determine whether the target functional domain is enriched with target biomarkers. The term “enriched” indicates that the target biomarkers appear in the target functional domain in a probability higher than the background or control level distribution of the biomarkers of the test sample. An enriched target functional domain shall contain enriched target biomarkers. Any suitable method known in the art may be used to determine whether a target functional domain is enriched.

In an illustrative embodiment, the probability of appearance of the target biomarkers in the target functional domain is calculated using the hypergeometric test. To perform the test, the biomarkers of the test sample are also grouped into functional domains according to the same classification criteria as the target functional domains. The functional domains of the biomarkers of the test sample are referred to as the test functional domains. In an illustrative embodiment, the biomarkers of the test sample that are genes functioning in cell cycle regulation are put into test functional domain tMᵢ; the biomarkers that are genes functioning in development are put into test functional domain tMⱼ. Therefore, to calculate the probability of appearance of the target biomarkers in target functional domain Mᵢ using the hypergeometric test, the following parameters are needed: (i) the number of target biomarkers in the target functional domain Mᵢ, (ii) the total number of target biomarkers in the test sample, (iii) the number of biomarkers that are grouped into the test functional domain tMᵢ; and (iv) the total number of biomarkers of the test sample. The biomarkers of the test sample are the tested biomarkers whose characteristics have been tested in the test sample. The calculation method of the hypergeometric test will be further described in the illustrative embodiment below. Other methods may be used for determining the probability of appearance of the target biomarkers, for example, Fisher's exact test (Fisher et al, On the interpretation of χ² from contingency tables, and the calculation of P, Journal of the Royal Statistical Society, 85(1):87-94, (1922)), gene set enrichment analysis (Subramanian et al, Gene set enrichment analysis: A knowledge-based approach for interpreting genome-wide expression profiles, PNAS, 102 (43):15545-15550, (2005)).

In certain embodiments, the probability of appearance of the target biomarkers in an enriched target functional domain has statistical significance. The level of statistical significance may be determined by a person practicing the method in accordance with the actual circumstances. In an illustrative embodiment, the level of statistical significance requires that the p-value is less than 0.01, or less than 0.05.

Flowing from the grouping operation 103, the processing of the method goes to the reference identification operation 105 as shown in FIG. 1. In the reference identification operation 105, the target biomarkers in target functional domains are used to identify one or more reference samples, wherein the reference samples have one or more reference biomarkers that show relevance to the one or more target biomarkers of the target functional domains. One or more characteristics of one or more biomarkers of the reference samples are measured and/or identified with or without the reference samples being contacted with the reference agents or being in the reference conditions. Changes in the one or more characteristics of the one or more biomarkers of a reference sample after the reference sample is contacted with a reference agent or get in a reference condition are obtained. The changes in the one or more characteristics of one or more target biomarkers in a target functional domain are compared with changes in the one or more characteristics of one or more biomarkers of a reference sample to determine whether these changes have relevance. The test sample and the reference sample shall share one or more common features in the types of their biological materials, biomarkers, and/or biomarker characteristics tested in order for the comparison of the two samples to be meaningful. The term “common feature” shall be construed broadly and can be any feature shared by both the test sample and the reference sample that would make the two samples comparable by a person with ordinary skill in the art.

The test sample and the reference sample may share some common features in their biological materials. In an illustrative embodiment, they are both breast cancer tissues. In another illustrative embodiment, the test sample is breast cancer tissue, the reference sample is lung cancer tissue but they are both cancer tissues. In another illustrative embodiment, the test sample is endothelial cells from human blood vessels, the reference sample is epithelial cells from mouse intestines, but they are both epithelium tissue. The test sample and reference sample may share some common features in the biomarkers and biomarker characteristics tested. In an illustrative embodiment, data for the test sample and the reference sample represents mRNA levels. In another illustrative embodiment, data for the test sample represents mRNA levels, and data for the reference sample represents protein levels, both represent gene expression levels.

In certain embodiments, data representing the characteristics of the biomarkers of one or more reference samples may be compiled into a database. The database may be previously published or otherwise publicly available or may be constructed de novo, or a combination of available databases and newly constructed data. In an illustrative embodiment, the Connectivity Map reported by Lamb et al. is used as the database for the reference samples, which consists of over 7000 gene expression profiles of human cultured cell lines treated with 1309 distinct chemical molecules, each of the gene expression profile may be used as a reference sample (Lamb et al. Science, 2006, 313, p1929-1935; Lamb, Nature Reviews Cancer 7, p 54-60 (2007)).

The relevance of the characteristics of the target biomarkers in the target functional domains and the characteristics of the reference biomarkers of the reference samples may be assessed by any suitable method known in the art. In certain embodiments, the relevance is assessed by statistical analysis methods. In an illustrative embodiment, the relevance is determined by a non-parametric statistical method. Examples of a non-parametric statistical method include, without limitation, Anderson-Darling test, Cochran's Q, Friedman two-way analysis of variance by ranks, Kendall's tau, Kendall's W, Kolmogorov-Smirnov test, Kruskal-Wallis one-way analysis of variance by ranks, Kuiper's test, Mann-Whitney U test, Maximum parsimony for the development of species relationships using computational phylogenetics, median test, Pitman's permutation test, Rank products, Siegel-Tukey test, Spearman's rank correlation coefficient, Student-Newman-Keuls (SNK) test, Van Elteren stratified Wilcoxon Rank Sum Test, Wald-Wolfowitz runs test, and Wilcoxon signed-rank test (Wasserman, All of Nonparametric Statistics, Springer, ISBN: 0387251456 (2007); Gibbons et al., Nonparametric Statistical Inference, 4th Ed. CRC, ISBN: 0824740521 (2003)). The statistical analysis methods may be modified by persons skilled in the art to suit specific situations.

In another illustrative embodiment, the Kolmogorov-Smirnov test is used to determine the relevance of the characteristics of target biomarkers of the target functional domains and the characteristics of the reference biomarkers. The Kolmogorov-Smirnov test calculates a KS-score or an S-score for a target functional domain and a reference sample (the calculation method will be further described in the illustrative embodiment below). The statistical significance of the KS-score or S-score is then calculated to determine whether the KS-score or the S-score is statistically significant. The statistical significance may be calculated by any method known in the art, including, without limitation, Student's t-test (Press et al, Numerical recipes in c: the art of scientific computing, Cambridge University Press. p. 616. ISBN 0521431085 (1992)), Chi-square test (Greenwood et al., A guide to chi-squared testing. Wiley, New York, ISBN 047155779X (1996)), Fisher F-test (Lomax et al, Statistical concepts: a second course, p. 10, ISBN 0805858504 (2007)), Z-test (Sprinthall et al., Basic statistical analysis: seventh edition, Pearson Education Group (2003)), permutation test (Good et al, Permutation, parametric and bootstrap tests of hypotheses, 3rd ed., Springer, ISBN 038798898X (2005)), and random permutation test (Nichols et al., Nonparametric permutation tests for functional neuro imaging: a primer with examples. Human Brain Mapping 15: 1-25, (2001)). When the KS-score or S-score of the target functional domain and the reference sample is statistically significant, the one or more target biomarkers of the target functional domain are considered relevant to the one or more reference biomarkers of the reference sample.

When the test sample has a plurality of target functional domains, the relevance calculation may be repeated for each target functional domain with respect to a reference sample. On the other hand, when there are more than one reference sample, the relevance calculation may be repeated for each reference sample with respect to a target functional domain. In another word, the relevance calculation may be performed between the set of target biomarkers of every target functional domain of the test sample and the set of biomarkers of every reference sample.

In certain embodiments, the target biomarkers in a target functional domain may be divided into two (or more) groups. One group contains target biomarkers that have test data with values (e.g. numerical) larger than (or increased from) the control data, indicating that those target biomarkers are up-regulated. The other group contains target biomarkers that have test data with values (e.g. numerical) smaller than (or decreased from) the control data, indicating that these target biomarkers are down-regulated. The target biomarkers may be divided into the up-regulated and down-regulated groups before the target biomarkers are grouped into target functional domains, or the target biomarkers may be grouped into target functional domains first and then divided into the up-regulated and down-regulated groups. The reference identification operation 105 may be performed separately for the up-regulated and down-regulated groups of target biomarkers in each target functional domain of the test sample. The reference identification operation 105 may be performed for the target biomarkers of the up-regulated group and the reference biomarkers, and/or the target biomarkers of the down-regulated group and the reference biomarkers.

In certain embodiments, the target biomarkers in enriched target functional domains are used to identify one or more reference samples in the reference identification operation 105, wherein the reference samples have one or more reference biomarkers that show relevance to the one or more target biomarkers of the enriched target functional domains.

After determining the relevance of target biomarkers in target functional domains (or the up-regulated groups or the down-regulated groups of target biomarkers in target functional domains, or target biomarkers in enriched target functional domains, or the up-regulated groups or the down-regulated groups of target biomarkers in enriched target functional domains) and the reference samples, the numbers of target functional domains (or enriched target functional domains) that show relevance to the reference samples may be counted. Such number for a reference sample is referred to as the relevance score of the reference sample. In an illustrative embodiment, if the target biomarkers of a test sample are grouped into 100 target functional domains, 20 out of the 100 target functional domains show relevance to reference sample 1, then the relevance score of reference sample 1 is 20. In the same illustrative embodiment, if 15 out of the 100 target functional domains show relevance to reference sample 2, then the relevance score of reference sample 2 is 15. The relevance score may be used as an indication of functional relevance between the test sample and the reference samples. The reference samples may be ranked based on their relevance scores. A reference sample with a higher relevance score can be considered having more functional relevance to the test sample than a reference sample with a lower relevance score. In the foregoing illustrative embodiment, reference sample 1 can be considered having more relevance to the test sample than reference sample 2.

It may also be determined whether the target functional domains of a test sample are positively relevant to a reference sample or negatively relevant to a reference sample. Positive relevance suggests that the biological effects that the test agent/condition has on the test sample are similar to those that the reference agent/condition has on the reference sample, and negative relevance suggests that the biological effects that the test agent/condition has on the test sample are contrary to what the reference agent/condition has on the reference sample. If KS-scores or S-scores are calculated, positive KS-scores or S-scores would indicate positive relevance, and negative KS-scores or S-scores would indicate negative relevance. Of course, any other method known in the art may be used to calculate the positive or negative relevance.

The number of target functional domains of a test sample having positive relevance to a reference sample, and the number of target functional domains having negative relevance to the reference sample may be counted separately. The reference samples may be ranked according to the positive relevance score or negative relevance score or the sum of the two scores. A higher positive relevance score suggests that the reference sample is more positively correlated with the test sample in biological functions. A higher negative relevance score suggests that the reference sample is more negatively correlated with the test sample in biological functions. In the illustrative embodiment given before, a test sample has 100 target functional domains and is compared with reference samples 1 and 2 for relevance. The test sample has 20 target functional domains that show relevance to reference sample 1, of which 15 show positive relevance and 5 show negative relevance. Meanwhile, the test sample has 15 target functional domains that show relevance to reference sample 2, of which 7 show positive relevance and 9 show negative relevance. In that event, reference sample 1 may be considered more positively correlated in function with the test sample but reference sample 2 may be considered more negatively correlated in function with the test sample.

Flowing from the reference identification operation 105, the method processing goes to the assessing operation 107 as shown in FIG. 1. In the assessing operation 107, the biological effects of the test agent/condition on the test sample are assessed based on biological effects of one or more reference agents/conditions on the one or more reference samples. When one or more relevant reference samples are identified, the biological effects of the reference agents/conditions on the reference samples are retrieved and reviewed. The biological effects of the reference agents/conditions on the reference samples may be already known or separately obtained. The biological effects of the test agent/condition are assessed based on the biological effects of the reference agents/conditions on those reference samples. In certain embodiments, the reference samples are ranked in ascending or descending order of their relevance scores. The biological effects of the test agent/condition on the test sample are predicted and evaluated based on the biological effects of one or more reference agents/conditions on reference samples with high relevance scores, for example, the reference samples with the top 20 highest relevance scores, or the top 10 highest relevance scores, or the top 5 highest relevance scores.

In certain embodiments, the reference samples are ranked in order of their positive or negative relevance scores. The biological effects of the test agent/condition on the test sample may be predicted to be similar (or contrary/adverse) to the biological effects of the reference agents/conditions on one or more reference samples with high positive (or negative) relevance scores, for example, the reference samples with the top 20 highest positive (or negative) relevance scores, or the top 10 highest positive (or negative) relevance scores, or the top 5 highest positive (or negative) relevance scores.

In an illustrative embodiment, the top 10 ranked reference samples calculated as described in Example 1 herein are listed in Table 1. The reference samples are ranked by their total relevance scores which are the sums of the positive relevance scores and the negative relevance scores, shown as “GO counts” in the table. The respective positive and negative relevance scores are shown in the parentheses. As shown in the table, the 10 reference samples all have positive relevance scores, and only the first reference sample on the list has a negative relevance score. The reference agents that the reference samples are contacted with are shown as “molecule” in the table. The test sample is acute myeloblastic leukemia cells OCI/AML2 treated with valproic acid. The table shows that three reference samples treated with valproic acid at different concentrations are among the top 10 ranked reference samples. The other top ranked reference samples are treated with trichostatin A, vorinostat and HC toxin, respectively, which are all histone deacetylase inhibitors, similar to the function of valproic acid. The results in Table 1 showed that the analysis methods described in this disclosure can identify reference samples treated with reference agents with similar biological functions. Similarly, if a compound x is used to treat the test sample in this illustrative embodiment and the same results as shown in Table 1 are obtained, it may be predicted that compound x has functions similar to the reference agents trichostatin A, valproic acid, vorinostat, HC toxin and ikarugamycin.

The reference agents may be analyzed for their common functional features and evaluate whether the test compound may have these features as well. The common functional features may include, without limitation, biological or physiological properties of the compounds, underlying biological mechanisms directly or indirectly affected by the compounds, physiological effects caused by the compounds, binding targets of the compounds and functional and structural similarity of the binding targets, metabolic products of the compounds and their functions, etc. The functional features of different reference agents may be given different weights in assessing the functions of the test compound depending on other factors, such as the positive relevance scores and the negative relevance scores, other information known about the reference agents and/or the test compound.

An illustrative embodiment of a method for assessing the biological effects of a test agent/condition as described in the present disclosure is disclosed below for illustration purpose only, and is not intended to limit the scope of the present disclosure in any way.

### Illustrative Embodiment

In this embodiment, gene expression (mRNA) of the test sample is measured using microarrays containing a plurality of gene probes. If there is more than one gene probe on the microarray that can bind to the same gene fragment, the amount of the gene may be calculated as the mean value of the amounts obtained from the multiple gene probes that can bind to that the same gene fragment. A set of test data representing gene expression of the test sample contacted with a test agent or in a test condition, and a set of control data representing gene expression of the test sample not contacted with a test agent nor in a test condition are obtained, respectively. The set of test data and the set of control data each comprise a plurality of data points representing the amounts of gene expression of the test sample. The test data and the control data are analyzed to identify the target gene/biomarker of the test sample.

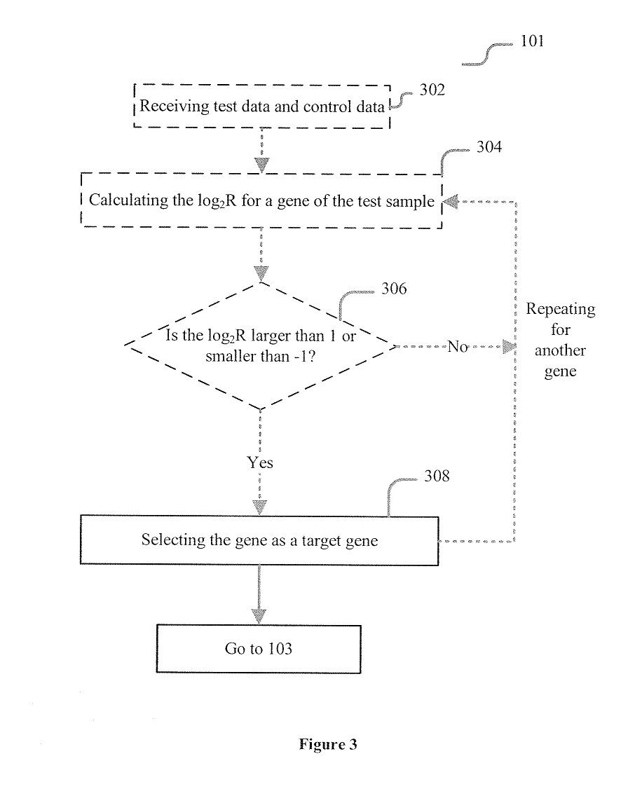

FIG. 3 shows an illustrative embodiment of the target identification operation 101, including an optional operation 302, that includes receiving test data and control data; an optional operation 304, that includes calculating the log₂R for a gene of the test sample; an optional decision operation 306, that includes checking whether the log₂R is larger than 1 or smaller than −1; and an operation 308, that includes selecting a gene as target gene. In the optional operation 302, a set of test data and a set of control data are received, respectively. The changes in gene expression of the test sample are calculated in the optional operation 304. For each gene, the change in gene expression is calculated as the log₂ of the ratio (i.e. log₂R) of the test data to the control data of the gene. Some of the genes show increase in gene expression after the test sample is contacted with the test agent or get in the test condition. For these genes, the log₂R is larger than zero. Some of the genes show decrease in gene expression after the test sample is contacted with the test agent or get in the test condition. For these genes, the log₂R is smaller than zero. In the optional decision operation 306 and operation 308, a gene is identified as a target gene if its log₂R is larger than 1 in the event of increase in gene expression, or smaller than −1 in the event of decrease in gene expression. The operations of optional operation 304, optional decision operation 306 and operation 308 are repeated for each gene for which test data and control data have been received until all of the target genes are identified.

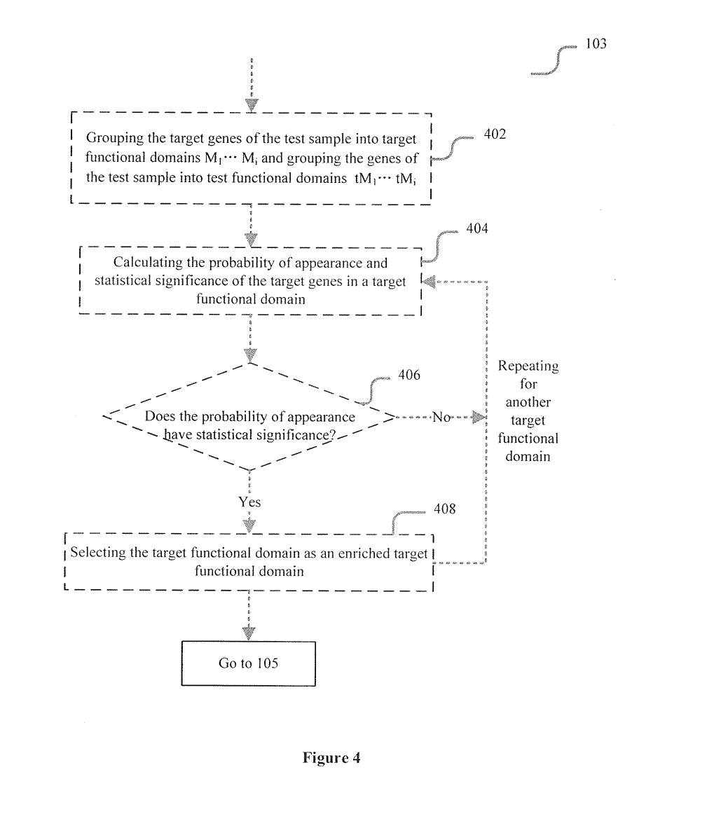

After the target genes of the test sample are identified, the target genes are grouped into one or more target functional domains, and then target functional domains are evaluated to determine whether they are enriched or not. FIG. 4 shows an illustrative embodiment of the grouping operation 103, including an optional operation 402, that includes grouping the target genes of the test sample into target functional domains M₁ . . . Mᵢ and grouping the genes of the test sample into test functional domains tM₁ . . . tMᵢ; an optional operation 404, that includes calculating the probability of appearance and statistical significance of the target genes in a target functional domain; an optional decision operation 406, that includes checking whether the probability of appearance of the target genes in a target functional domain have statistical significance (“Yes” means having statistical significance and “No” means not having statistical significance); and an optional operation 408, that includes selecting the target functional domain as an enriched target functional domain if the decision operation returns “Yes”.

The operation begins with optional operation 402, in which, the target genes are grouped into one or more target functional domains M₁ . . . Mᵢ according to gene ontology classification rules, and the tested genes (i.e. the genes for which gene expression data has been measured) of the test sample are grouped into one or more test functional domains tM₁ . . . tMᵢ according to the gene ontology classification rules. A test functional domain corresponds with a target functional domain if they contain genes in the same functional group. In an illustrative embodiment, a test functional domain that contains genes functioning in cell cycle regulation corresponds with a target functional domain that also contains genes functioning in cell cycle regulation.

Flowing from the optional operation 402, the processing goes to the optional operation 404. In the optional operation 404, the number of target genes in a target functional domain Mᵢ is compared with the number of genes in the corresponding test functional domain tMᵢ to determine whether the appearance of the target genes in target functional domain Mᵢ is a statistically significant event. The hypergeometric test is applied to calculate the probability of appearance of the target genes in the target functional domain Mᵢ and the p-value of the probability of appearance. The hypergeometric test comprises calculating the probability of appearance and the p-value using the following Equations 1 and 2:

$$\begin{matrix}f ( k , N , m , n ) = \frac{( \begin{matrix}m \\ k\end{matrix} ) ( \begin{matrix}N - m \\ n - k\end{matrix} )}{( \begin{matrix}N \\ n\end{matrix} )} ; & ( Equation 1 ) \\ P ( k ) = P ( x \geq k ) = 1 - {\sum }_{x = 0}^{k - 1} f ( x , N , m , n ) ; & ( Equation 2 )\end{matrix}$$

wherein f(k, N, m, n) is the probability of appearance of a total of k target genes in the target functional domain Mᵢ, and P(k) is the p-value for the target functional domain Mᵢ; N represents the total number of tested genes of the test sample, m represents the number of tested genes of the test functional domain tMᵢ, n represents the total number of target genes of the test sample, k represents the number of target genes of the target functional domain Mᵢ.

The statistical significance of the probability of appearance of the target genes is represented by the p-value. If the p-value is less than 0.01, then the probability of appearance is considered having statistical significance. The statistical significance may also be set at p-value<0.05. A target functional domain in which the probability of appearance of the number of target genes is statistically significant, is selected as an enriched target functional domain in the optional decision operation 406 and the optional operation 408. The analysis process is repeated for each target functional domain of the test sample until all of the enriched target functional domains are determined.

Then the enriched target functional domains are used to identify one or more reference samples from a reference database. The reference database is obtained from the Connectivity Map reported by Lamb et al. (Lamb et al. Science, 2006, 313, p 1929-1935). The reference database may be accessed through the storage path of the reference database recorded on a storage medium. The reference database comprises a plurality of reference samples, wherein each reference sample comprises a plurality of data points representing gene expression of the reference sample that is contacted with a reference agent or in a reference condition in which the biological effects of the reference agent/condition on the reference sample are known.

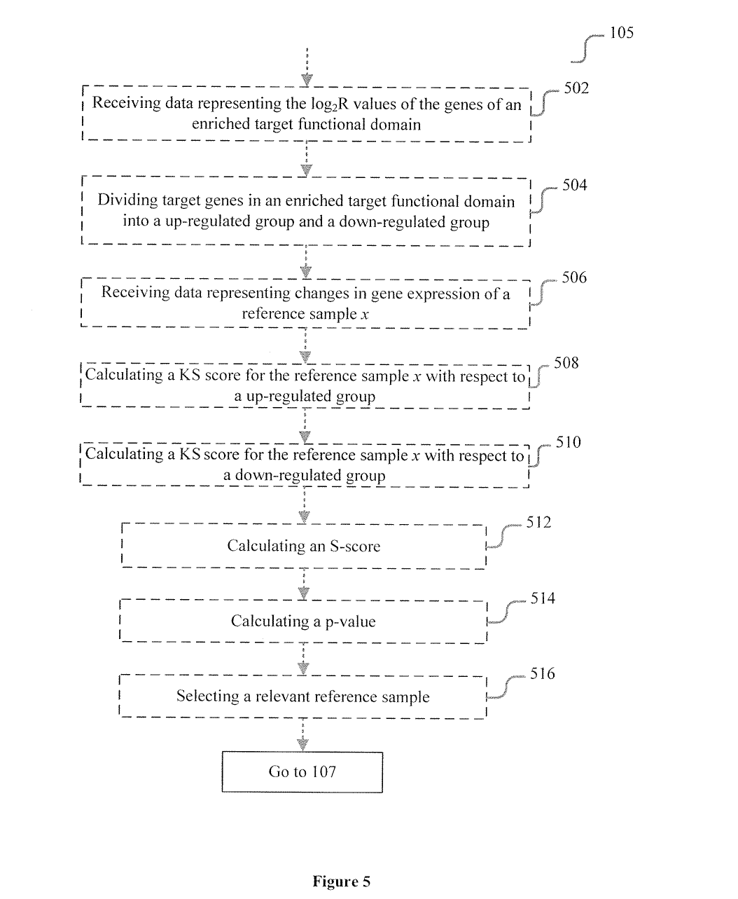

FIG. 5 shows an illustrative embodiment of the reference identification operation 105, including an optional operation 502, that includes receiving data representing the log₂R values of the genes of an enriched target functional domain; an optional operation 504, that includes dividing target genes in an enriched target functional domain into up-regulated and down-regulated groups; an optional operation 506, that includes receiving data representing changes in gene expression of a reference sample x, an optional operation 508, that includes calculating a KS score for the reference sample x with respect to the up-regulated group; an optional operation 510, that includes calculating a KS score for the reference sample x with respect to the down-regulated group; an optional operation 512, that includes calculating an S-score; an optional operation 514, that includes calculating a p-value; and an optional operation 516, that includes selecting a relevant reference sample. In certain embodiments, the operations in FIG. 5 may be performed in different orders or repeated in whole or in part. In an illustrative embodiment, the optional operations 502 and 506 are performed concurrently. In another illustrative embodiment, the optional operation 506 is performed before the optional operation 502. In another illustrative embodiment, the optional operations 508 and 510 are performed concurrently. In yet another illustrative embodiment, the optional operation 510 is performed before the optional operation 508.

In the optional operation 502, data representing the log₂R values of the genes of an enriched target functional domain is received. The target genes in an enriched target functional domain are divided into two groups in the optional operation 504: the up-regulated and the down-regulated group. In the optional operation 506, data representing changes in gene expression of a reference sample x is received. In the optional operation 508 and the optional operation 510, the relevance of the target genes of the enriched target functional domain and the genes of reference sample x are assessed by the Kolmogorov-Smirnov test (K-S test). The KS scores are calculated respectively for the up-regulated group and the down-regulated group of target genes of the enriched target functional domain using the Equations 3-5 below:

$$\begin{matrix}a = {Max}_{j = 1}^{t} [ \frac{W ( j )}{t} - \frac{V ( j )}{N} ] ; & ( Equation 3 ) \\ b = {Max}_{j = 1}^{t} [ \frac{V ( j )}{N} - \frac{[ W ( j ) - 1 ]}{t} ] ; & ( Equation 4 ) \\ K S score = \{ \begin{matrix}a , & ( a > b ) \\ - b , & ( b > a ) ;\end{matrix} & ( Equation 5 )\end{matrix}$$

wherein tis the number of target genes in the up-regulated group (or the down-regulated group); j is the jth target gene in the up-regulated group (or the down-regulated group); W(j) is the rank of target gene j among the target genes in the up-regulated group (or the down-regulated group) based on the change in its gene expression; V(j) is the rank of the reference gene j, which is the same or corresponding gene as the target gene j, among the reference genes of reference sample x based on the change in its gene expression; and N is the total number of tested reference genes in reference sample x. Corresponding genes may be determined in any suitable methods that those skilled in the art may use. In an illustrative embodiment, a corresponding reference gene has exactly the same sequence as the target gene. In another illustrative embodiment, a corresponding reference gene is the counterpart of the target gene in a difference species. In another illustrative embodiment, a corresponding reference gene is a mutant or variant of the target gene.

In the optional operation 512, the S-score for the enriched target functional domain and reference sample x is calculated using the following Equation 6:

$$S \text{-} score = \{ \begin{matrix}{KS}_{up} - {KS}_{down} , & ( {KS}_{up} \times {KS}_{down} < 0 ) \\ 0 , & ( {KS}_{up} \times {KS}_{down} \geq 0 ) ,\end{matrix} \qquad ( Equation 6 )$$

wherein the KSᵤₚ is the KS score for the up-regulated group, KS_{down} is the KS score the down-regulated group.

If the S-score is zero, it is considered not statistically significant. If the S-score is not zero, the p-value of the S-score is calculated to determine the statistical significance of the S-score in the optional operation 514. In this illustrative embodiment, the permutation method is applied to calculate the p-value of each S-score. The permutation method comprises the following calculations: (a) calculating a plurality of hypothetical KSᵤₚ and KS_{down} scores using Equations 3-5 above, based on randomly ranked genes in reference sample x; (b) calculating hypothetical S-scores based on the hypothetical KSᵤₚ and KS_{down} scores using Equation 6 above; (c) calculating the percentage of times when the absolute value of a hypothetical S-score is higher than the value of the S-score of the enriched target functional domain, and this percentage is the p-value for the S-score of the target genes in the enriched target functional domain. Absolute value describes the distance of a number on the number line from 0 without considering which direction from zero the number lies. The absolute value of a number is never negative. In an illustrative embodiment, the absolute values of 1 or −1 are both 1.

To calculate the hypothetical S-score, the genes in reference sample x are randomly ranked in orders. The hypothetical KS scores and hypothetical S-score of the enriched target functional domain of test sample are calculated using Equations 3-6. A hypothetical KSᵤₚ score (hKSᵤₚ) is obtained for the up-regulated group, wherein W(j) is the rank of target gene I among all target genes in the up-regulated gene group based on the change in gene expression of target gene j, V(j) is the rank of gene j in the randomly permutated genes in reference sample x. A hypothetical KS_{down} score (hKS_{down}) is obtained for the down-regulated group by the same method. A hypothetical S-score (hS-score) for the enriched target functional domain may be calculated using the following equation:

$$hS \text{-} score = \{ \begin{matrix}{hKS}_{up} - {hKS}_{down} , & ( {hKS}_{up} \times {hKS}_{down} < 0 ) \\ 0 , & ( {hKS}_{up} \times {hKS}_{down} \geq 0 ) .\end{matrix}$$

The calculation of the hypothetical S-score is repeated for 1000 times wherein the genes in reference sample x are randomly ranked in orders for each calculation of a hypothetical S-score. The absolute values of the hypothetical S-scores so obtained are compared with that of the actual S-score of the enriched target functional domain to determine whether the absolute values of the hypothetical S-score are higher than that of the actual S-score. The percentage of times when the absolute values of the hypothetical S-scores are higher than the actual S-score is calculated as the p-value of the S-score. In this illustrative embodiment, the level of statistical significance requires that the p-value is less than 0.05. The statistical significance may also be set at p-value<0.01.

The S-score and p-value are calculated for each enriched target functional domains with respect to reference sample x. Reference sample x may be considered having relevance to the test sample if reference sample x has at least one, five or ten statistically significant S-scores with respect to a test sample.

In this illustrative embodiment, the reference database comprises more than one reference sample, therefore S-scores are calculated for each reference sample with respect to each enriched target functional domains of the test sample. Optionally, the number of statistically significant S-scores for each reference sample is counted, and the count is used as an indication of functional relevance between the test sample and the reference sample. The reference samples may be ranked based on the count of statistically significant S-scores that they have for the test sample. The reference sample with a higher count may be considered having more functional relevance to the test sample than a reference sample with a lower count.

In this illustrative embodiment, optionally, the total number of positive S-scores and the total number of negative S-scores are counted with respect to each reference sample. Positive S-scores suggest that the biological effects that the test agent/condition has on the test sample are similar to those that the reference agent/condition has on the reference sample, and negative S-scores indicate that the biological effects that the test agent/condition has on the test sample are contrary to those that the reference agent/condition has on the reference sample.

The biological effects of the test agent/condition are assessed based on the biological effects of the reference agents/conditions on the one or more reference samples. The reference samples that have the top 10 highest relevance scores (including both positive relevance scores and negative relevance scores) are used to assess the function of the test agent/condition. Information regarding the reference agents/conditions on the reference samples is reviewed and analyzed. Common features among the biological functions and features of the reference agents/conditions are identified and evaluated. The 10 reference samples are re-ranked based on the positive relevance scores and the negative relevance scores, respectively, the biological functions and features of the reference agents/conditions are re-evaluated based on the new rankings. The biological functions and features of the test agent/condition are predicted and inferred from the functions and features of the reference agents/conditions.

Computer Program Product, Computer Readable Storage Medium and Computer System

In certain embodiments, the present disclosure provides a computer program product comprising one or more instructions recorded on a machine-readable recording medium for assessing the biological effects of a test agent/condition, wherein the one or more instructions comprise: one or more instructions for identifying one or more target biomarkers from a test sample contacted with the test agent or in the test condition; one or more instructions for grouping the one or more target biomarkers into one or more target functional domains according to pre-determined criteria; one or more instructions for identifying one or more reference samples having relevance to the test sample; one or more instructions for assessing the biological effects of the test agent/condition based on the biological effects of the one or more reference agents/conditions on the one or more reference samples; and one or more instructions for outputting the assessing results.

A person with ordinary skill in the art will appreciate that a computer program product described herein are capable of being distributed in a variety of forms via a signal bearing medium, and that the program product described herein applies regardless of the particular type of signal bearing medium used to actually carry out the distribution. Examples of a signal bearing medium include, but are not limited to, the following: a recordable type medium such as a floppy disk, a hard disk drive, a Compact Disc (CD), a Digital Video Disk (DVD), a digital tape, a computer memory, etc.; and a transmission type medium such as a digital and/or an analog communication medium (e.g., a fiber optic cable, a waveguide, a wired communications link, a wireless communication link, etc.).

In certain embodiments, the one or more instructions for identifying one or more target biomarkers comprises: one or more instructions for receiving a set of test data representing one or more characteristics of one or more biomarkers of the test sample contacted with the test agent or in the test condition and a set of control data representing the characteristics of one or more biomarkers of the test sample not contacted with the test agent or in the test condition; one or more instructions for calculating changes between the test data and the control data for the one or more biomarkers; and one or more instructions for selecting one or more target biomarkers of the test sample, wherein each target biomarker shows changes between the test data and the control data.

In certain embodiments, the one or more instructions for grouping the one or more target biomarkers into one or more target functional domains further comprises one or more instructions for identifying one or more enriched target functional domains.

In certain embodiments, the one or more instructions for identifying one or more enriched target functional domains comprises: one or more instructions for calculating the probability of appearance of the one or more target biomarkers in a target functional domain of the test sample; one or more instructions for calculating the statistical significance of said probability of appearance; one or more instructions for repeating the above calculation of the probability of appearance and the statistical significance for each target functional domains of the test sample; and one or more instructions for selecting one or more enriched target functional domains.

In certain embodiments, the probability of appearance and the statistical significance of the one or more target biomarkers are determined according to Equations 1 and 2 described herein.

In certain embodiments, the one or more instructions for identifying one or more reference samples comprises: one or more instructions for calculating the KS score for the one or more target biomarkers of a target functional domain of the test sample with respect to a reference sample according to Equations 3-5 described herein; one or more instructions for determining the statistical significance of the above calculated KS score; one or more instructions for repeating the above calculation of KS score and determination of statistical significance for each target functional domains of the test sample with respect to every reference sample; and one or more instructions for selecting the reference samples that have at least one statistically significant KS score.

In certain embodiments, the statistical significance of the KS score is represented by the p-value calculated as the percentage of times when the absolute value of a hypothetical KS score is higher than the absolute value of the KS score, and wherein the hypothetical KS score is calculated using the K-S Test based on randomly ranked reference biomarkers of the reference sample.

In certain embodiments, the one or more instructions for identifying one or more reference samples further comprises: one or more instructions for counting the number of target functional domains that have statistically significant KS scores with respect to every reference sample; and one or more instructions for ranking the reference samples based on their numbers of statistically significant KS scores.

In certain embodiments, the one or more instructions for identifying one or more reference samples comprises: one or more instructions for calculating the KS score for the one or more target biomarkers of an enriched target functional domain of the test sample with respect to a reference sample according to Equations 3-5 described herein; one or more instructions for determining the statistical significance of the above calculated KS score; one or more instructions for repeating the above calculation of KS score and determination of statistical significance for each enriched target functional domains of the test sample with respect to every reference sample; and one or more instructions for selecting the reference samples that have at least one statistically significant KS score.

In certain embodiments, the one or more instructions for identifying one or more reference samples further comprises: one or more instructions for counting the number of enriched target functional domains that have statistically significant KS scores with respect to every reference sample; and one or more instructions for ranking the reference samples based on their numbers of statistically significant KS scores.

In certain embodiments, the one or more instructions for identifying one or more reference samples comprises: one or more instructions for separating the one or more target biomarkers of a target functional domain of the test sample into an up-regulated group and a down-regulated group; one or more instructions for calculating a KS score for the up-regulated group and a KS score for the down-regulated group with respect to a reference sample according to Equations 3-5 described herein; one or more instructions for calculating the S-score for the target functional domain according to Equation 6 described herein; one or more instructions for calculating the p-value of the S-score of the target functional domain; one or more instructions for repeating the above calculation of S-score and p-value for each target functional domains of the test sample with respect to every reference sample; and one or more instructions for selecting the reference samples that have at least one statistically significant S-score.

In certain embodiments, the one or more instructions for identifying one or more reference samples further comprises: one or more instructions for counting the number of statistically significant S-scores for the test sample with respect to every reference sample; and one or more instructions for ranking the reference samples based on their numbers of statistically significant S-scores.

In certain embodiments, the one or more instructions for identifying one or more reference samples comprises: one or more instructions for separating the one or more target biomarkers of an enriched target functional domain of the test sample into an up-regulated group and a down-regulated group; one or more instructions for calculating a KS score for the up-regulated group and a KS score for the down-regulated group with respect to a reference sample according to Equations 3-5 described herein; one or more instructions for calculating the S-score for the enriched target functional domain according to Equation 6 described herein; one or more instructions for calculating the p-value of the S-score of the enriched target functional domain; one or more instructions for repeating the above calculation of S-score and p-value for each enriched target functional domains of the test sample with respect to every reference sample; and one or more instructions for selecting the reference samples that have at least one statistically significant S-score.

In certain embodiments, the one or more instructions for assessing the biological effects comprises: one or more instructions for retrieving the biological effects of the one or more reference agents/conditions on the one or more relevant reference samples; and one or more instructions for assessing the biological effects of the test agent/condition based on the biological effects of the one or more reference agents/conditions.

In certain embodiments, the present disclosure provides a computer readable storage medium having a computer program encoded thereon, said computer program when executed by a computer system instructs the computer system to execute a method for assessing biological effects of a test agent/condition, which comprises: identifying one or more target biomarkers from a test sample contacted with the test agent or in the test condition; grouping the one or more target biomarkers into one or more target functional domains according to pre-determined criteria; identifying one or more reference samples having relevance to the test sample; assessing the biological effects of the test agent/condition based on the biological effects of the one or more reference agents/conditions on the one or more reference samples, and outputting the assessing results.

In certain embodiments, said identifying one or more target biomarkers comprises: receiving a set of test data representing one or more characteristics of one or more biomarkers of the test sample contacted with the test agent or in the test condition, and a set of control data representing the characteristics of one or more biomarkers of the test sample not contacted with the test agent or in the test condition; calculating changes between the test data and the control data for the one or more biomarkers; and selecting one or more target biomarkers of the test sample, wherein each target biomarker shows changes between the test data and the control data.

In certain embodiments, said grouping the one or more target biomarkers into one or more target functional domains further comprises identifying one or more enriched target functional domains.

In certain embodiments, said identifying one or more enriched target functional domains comprises: calculating the probability of appearance of the one or more target biomarkers in the target functional domain; calculating the statistical significance of said probability of appearance; repeating the above calculation of the probability of appearance and the statistical significance for each target functional domains of the test sample; and selecting one or more enriched target functional domains.

In certain embodiments, the probability of appearance and the statistical significance of the one or more target biomarkers are determined according to the Equations 1 and 2 described herein.

In certain embodiments, said identifying one or more reference samples comprises: for the one or more target biomarkers in a target functional domain, calculating the KS score for the one or more target biomarkers with respect to a reference sample according to the Equations 3-5 described herein; determining the statistical significance of the above calculated KS score; repeating the above calculation of KS score and determination of statistical significance for each target functional domains of the test sample with respect to every reference sample; and selecting the reference samples that have at least one statistically significant KS score.

In certain embodiments, the statistical significance of the KS score is represented by the p-value calculated as the percentage of times when the absolute value of a hypothetical KS score is higher than the absolute value of the KS score, and wherein the hypothetical KS score is calculated using the K-S Test based on randomly ranked reference biomarkers of the reference sample.

In certain embodiments, said identifying one or more reference samples further comprises: counting the number of target functional domains that halve statistically significant KS scores with respect to every reference sample; and ranking the reference samples based on their numbers of statistically significant KS scores.

In certain embodiments, said identifying one or more reference samples comprises: for the one or more target biomarkers in an enriched target functional domain, calculating the KS score for the one or more target biomarkers with respect to a reference sample according to the Equations 3-5 described herein; determining the statistical significance of the above calculated KS score; repeating the above calculation of KS score and determination of statistical significance for each enriched target functional domains of the test sample with respect to every reference sample; and selecting the reference samples that have at least one statistically significant KS score.

In certain embodiments, said identifying one or more reference samples further comprises: counting the number of enriched target functional domains that have statistically significant KS scores with respect to every reference sample; and ranking the reference samples based on their numbers of statistically significant KS scores.

In certain embodiments, said identifying one or more reference samples comprises: for the one or more target biomarkers in a target functional domain, separating the one or more target biomarkers into an up-regulated group and a down-regulated group; calculating a KS score for the up-regulated group and a KS score for the down-regulated group with respect to a reference sample according to the Equations 3-5 described herein; calculating the S-score for the target functional domain according to the Equation 6 described herein; calculating the p-value of the S-score of the target functional domain; repeating the above calculation of S-score and p-value for each target functional domains of the test sample with respect to every reference sample; and selecting the reference samples that have at least one statistically significant S-score.

In certain embodiments, said identifying one or more reference samples further comprises: counting the number of statistically significant S-scores for the test sample with respect to every reference sample; and ranking the reference samples based on their numbers of statistically significant S-scores.

In certain embodiments, said identifying one or more reference samples comprises: for the one or more target biomarkers in an enriched target functional domain, separating the one or more target biomarkers into an up-regulated group and a down-regulated group; calculating a KS score for the up-regulated group and a KS score for the down-regulated group with respect to a reference sample according to the Equations 3-5 described herein; calculating the S-score for the enriched target functional domain according to the Equation 6 described herein; calculating the p-value of the S-score of the enriched target functional domain; repeating the above calculation of S-score and p-value for each enriched target functional domains of the test sample with respect to every reference sample; and selecting the reference samples that have at least one statistically significant S-score.

In certain embodiments, said assessing the biological effects comprises: retrieving the biological effects of the one or more reference agents or reference conditions on the one or more identified reference samples; and assessing the biological effects of the test agent or the test condition based on the biological effects of the one or more reference agents or reference conditions.

In certain embodiments, the computer readable storage medium may be any of a variety of memory storage devices. Examples of memory storage medium include any commonly available random access memory (RAM), magnetic medium such as a resident hard disk or tape, an optical medium such as a read and write compact disc, or other memory storage device.

In certain embodiments, the present disclosure provides a system comprising one or more input devices, one or more output devices, one or more processors, and one or more memory devices storing therein one or more operating systems, one or more computer programs, and one or more optional databases, interconnected by a bus. The one or more computer programs are executable by the system. The one or more processors are instructed by the one or more computer programs to execute a method for assessing biological effects of a test agent/condition on a test sample. The one or more computer programs comprise: one or more instructions to cause the one or more processors to identify one or more target biomarkers from a test sample contacted with the test agent or in the test condition, one or more instructions to cause the one or more processors to group the one or more target biomarkers into one or more target functional domains according to pre-determined criteria; one or more instructions to cause the one or more processors to identify one or more reference samples having relevance to the test sample; one or more instructions to cause the one or more processors to assess the biological effects of the test agent/condition based on the biological effects of the one or more reference agents/conditions on the one or more reference samples; and one or more instructions to cause the one or more processors to output the assessing results.

In certain embodiments, the one or more instructions to cause the one or more processors to identify one or more target biomarkers comprise: one or more instructions to cause the one or more processors to receive a set of test data representing one or more characteristics of one or more biomarkers of the test sample contacted with the test agent or in the test condition, and a set of control data representing the characteristics of one or more biomarkers of the test sample not contacted with the test agent or in the test condition; one or more instructions to cause the one or more processors to calculate changes between the test data and the control data for the one or more biomarkers; and one or more instructions to cause the one or more processors to select one or more target biomarkers of the test sample, wherein each target biomarker shows changes between the test data and the control data.

In certain embodiments, said one or more instructions to cause the one or more processors to identify one or more target functional domains further comprises identifying one or more enriched target functional domains.

In certain embodiments, said one or more instructions to cause the one or more processors to identify one or more enriched target functional domains comprise: one or more instructions to cause the one or more processors to calculate the probability of appearance of the one or more target biomarkers in the target functional domain; one or more instructions to cause the one or more processors to calculate the statistical significance of said probability of appearance; one or more instructions to cause the one or more processors to repeat the above calculation of the probability of appearance and the statistical significance for each target functional domain of the test sample; and one or more instructions to cause the one or more processors to select one or more enriched target functional domains.

In certain embodiments, the probability of appearance and the statistical significance of the one or more target biomarkers are determined according to the Equations 1 and 2 described herein.

In certain embodiments, the one or more instructions to cause the one or more processors to identify one or more reference samples comprise: one or more instructions to cause the one or more processors to calculate the KS score for the one or more target biomarkers of a target functional domain of the test sample with respect to a reference sample according to the Equations 3-5 described herein; one or more instructions to cause the one or more processors to determine the statistical significance of the above calculated KS score; one or more instructions to cause the one or more processors to repeat the above calculation of KS score and determination of statistical significance for each target functional domains of the test sample with respect to every reference sample; and one or more instructions to cause the one or more processors to select the reference samples that have at least one statistically significant KS score.

In certain embodiments, the statistical significance of the KS score is represented by the p-value calculated as the percentage of times when the absolute value of a hypothetical KS score is higher than the absolute value of the KS score, and wherein the hypothetical KS score is calculated using the K-S Test based on randomly ranked reference biomarkers of the reference sample.

In certain embodiments, the one or more instructions to cause the one or more processors to identify one or more reference samples further comprise: one or more instructions to cause the one or more processors to count the number of target functional domains that have statistically significant KS scores with respect to every reference sample; and one or more instructions to cause the one or more processors to rank the reference samples based on their numbers of statistically significant KS scores.

In certain embodiments, the one or more instructions to cause the one or more processors to identify one or more reference samples comprise: one or more instructions to cause the one or more processors to calculate the KS score for the one or more target biomarkers of an enriched target functional domain of the test sample with respect to a reference sample according to the Equations 3-5 described herein; one or more instructions to cause the one or more processors to determine the statistical significance of the above calculated KS score; one or more instructions to cause the one or more processors to repeat the above calculation of KS score and determination of statistical significance for each enriched target functional domains of the test sample with respect to every reference sample; and one or more instructions to cause the one or more processors to select the reference samples that have at least one statistically significant KS score.

In certain embodiments, the one or more instructions to cause the one or more processors to identify one or more reference samples further comprise: one or more instructions to cause the one or more processors to count the number of enriched target functional domains that have statistically significant KS scores with respect to every reference sample; and one or more instructions to cause the one or more processors to rank the reference samples based on their numbers of statistically significant KS scores.

In certain embodiments, the one or more instructions to cause the one or more processors to identify one or more reference samples comprise: one or more instructions to cause the one or more processors to separate the one or more target biomarkers of a target functional domain of the test sample into an up-regulated group and a down-regulated group; one or more instructions to cause the one or more processors to calculate a KS score for the up-regulated group and a KS score for the down-regulated group with respect to a reference sample according to the Equations 3-5 described herein; one or more instructions to cause the one or more processors to calculate the S-score for the target functional domain according to the Equation 6 described herein; one or more instructions to cause the one or more processors to calculate the p-value of the S-score of the target functional domain; one or more instructions to cause the one or more processors to repeat the above calculation of S-score and p-value for each target functional domains of the test sample with respect to every reference sample; and one or more instructions to cause the one or more processors to select the reference samples that have at least one statistically significant S-score.

In certain embodiments, the one or more instructions to cause the one or more processors to identify the one or more reference samples further comprise: one or more instructions to cause the one or more processors to count the number of statistically significant S-scores for the test sample with respect to every reference sample; and one or more instructions to cause the one or more processors to rank the reference samples based on their numbers of statistically significant S-scores.

In certain embodiments, the one or more instructions to cause the one or more processors to identify the one or more reference samples comprise: one or more instructions to cause the one or more processors to separate the one or more target biomarkers of an enriched target functional domain of the test sample into an up-regulated group and a down-regulated group; one or more instructions to cause the one or more processors to calculate a KS score for the up-regulated group and a KS score for the down-regulated group with respect to a reference sample according to the Equations 3-5 described herein; one or more instructions to cause the one or more processors to calculate the S-score for the enriched target functional domain according to the Equation 6 described herein; one or more instructions to cause the one or more processors to calculate the p-value of the S-score of the enriched target functional domain; one or more instructions to cause the one or more processors to repeat the above calculation of S-score and p-value for each enriched target functional domains of the test sample with respect to every reference sample; and one or more instructions to cause the one or more processors to select the reference samples that have at least one statistically significant S-score.

In certain embodiments, the one or more instructions to cause the one or more processors to assess the biological effects comprise: one or more instructions to cause the one or more processors to retrieve the biological effects of the one or more reference agents or reference conditions on the one or more identified reference samples; and one or more instructions to cause the one or more processors to assess the biological effects of the test agent or the test condition based on the biological effects of the one or more reference agents or reference conditions.

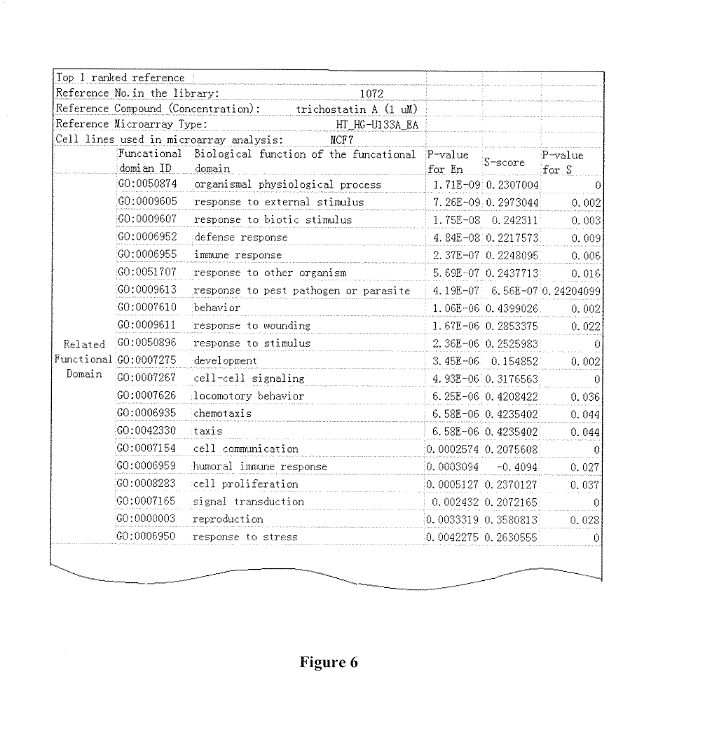

The analysis results including assessing results obtained by a method described herein may be output by the computer in any suitable form, including, without limitation, charts and graphs. In certain embodiments, the analysis results may be in the form of one or more data charts. The data chart may contain information relevant to target functional domains or enriched target functional domains such as names and functional characteristics of the target functional domains or enriched target functional domains, calculation results such as KS-scores, p-values, S-scores, ranking results of the reference samples, description of the potential biological effects of the test agents/conditions. An illustrative data chart is shown in FIG. 6. In FIG. 6, the chart shows the ID Numbers of the enriched target functional domains (in the column “Functional domain ID”), their functional characteristics (in the column “Biological function of the functional domain”), the p-value for the enriched target functional domains (in the column “p-value for En”), the S-scores for the enriched target functional domains (in the column “S-score”), and the p-value for the S-scores (in the column “p-value for S”).

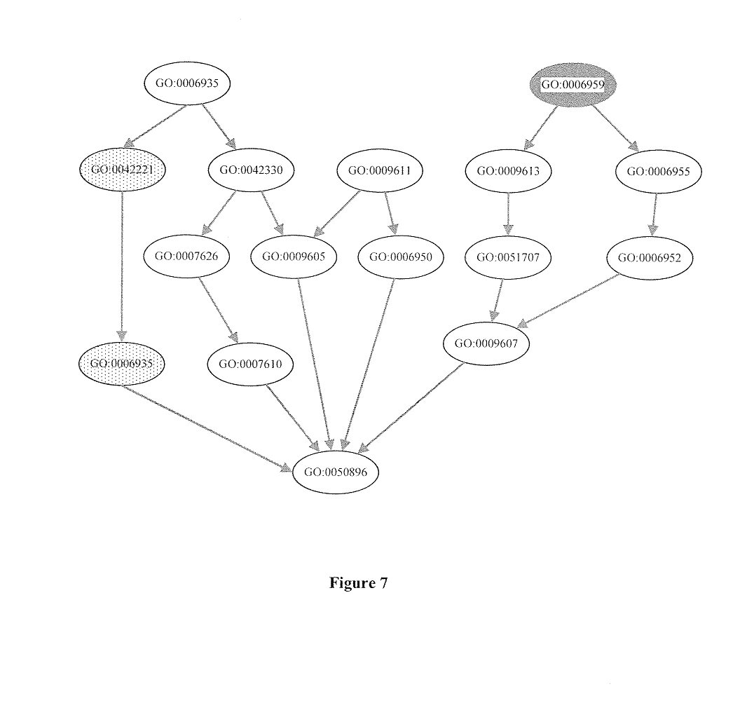

In certain embodiments, the analysis results may be displayed in the form of one or more graphs. The output graph may show the functional relationship among the one or more enriched target functional domains that show relevance to a reference sample. An illustrative example is shown in FIG. 7. In FIG. 7, a graph shows various circles representing various functional domains of the test sample classified according to the classification rules of gene ontology. The circles that are functionally related are connected by arrows. The empty circles represent functional domains that show positive relevance to the reference sample, the filled circles represent functional domains that show negative relevance to the reference sample, the dotted circles represent functional domains that show no relevance to the reference sample.

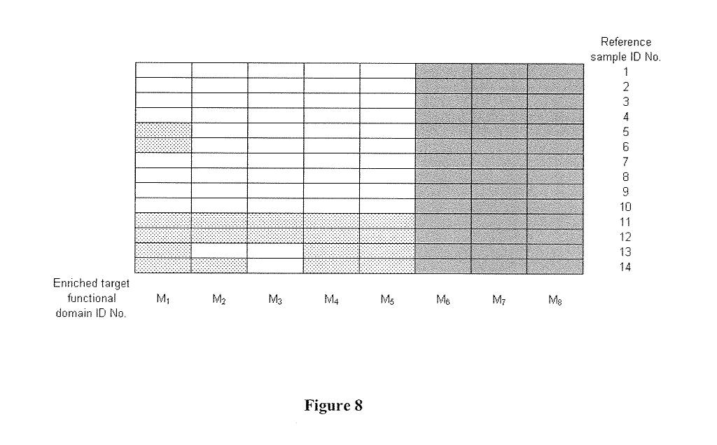

The computer output display may also separately show the relevance of the reference samples to the one or more enriched target functional domains of the test sample. An illustrative example is shown in FIG. 8. FIG. 8 shows a table in which the enriched target functional domains of the test sample are listed in the columns and the reference samples are listed in the rows. The relevance of each reference sample to each enriched target functional domain is shown in the cells of the table. An empty cell, filled cell, and dotted cell indicate that the relevance reference sample and the enriched target functional domain have positive relevance, negative relevance, and no relevance, respectively.

When a method provided herein is executed by a computer system, the output of the analysis results obtained from the method may be delivered through the one or more output device of the computer system, including but not limited to, a computer display screen and/or a printer, etc.

A processor used herein may include, without limitation, one or more microprocessors, field programmable logic arrays, or application specific integrated circuits. Illustrative processors include, but are not limited to, Intel Corp's Pentium series processors, Sun Microsystems' SPARC processors, Motorola Corp.'s PowerPC processors and Dragonball processors, MIPS Technologies Inc.'s MIPs processors, Xilinx Inc.'s Vertex series of field programmable logic arrays, and other processors.

An operating system used herein may comprise machine code that, once executed by a processor, coordinates and executes functions of other parts in a computer and facilitates the processor to execute the functions of various computer programs that may be written in a variety of programming languages. In addition to managing data flow among other parts in a computer, an operating system also provides scheduling, input-output control, file and data management, memory management, and communication control and related services, all in accordance with known techniques. Illustrative operating systems include, for example, Windows operating systems from the Microsoft Corporation, Unix or Linux-type operating systems available from many vendors, MAC operating systems from Apple, another or a future operating system, and some combination thereof.

A database used herein may include information about the biological features and functions of the one or more biomarkers of the test sample, information about the reference database including the biological features and functions of the biomarkers of the reference samples, the biological effects of the reference agents/conditions, and any other relevant information. Besides the database, the above mentioned information may also be inputted into the system through the input device from an external storage medium or through a network.

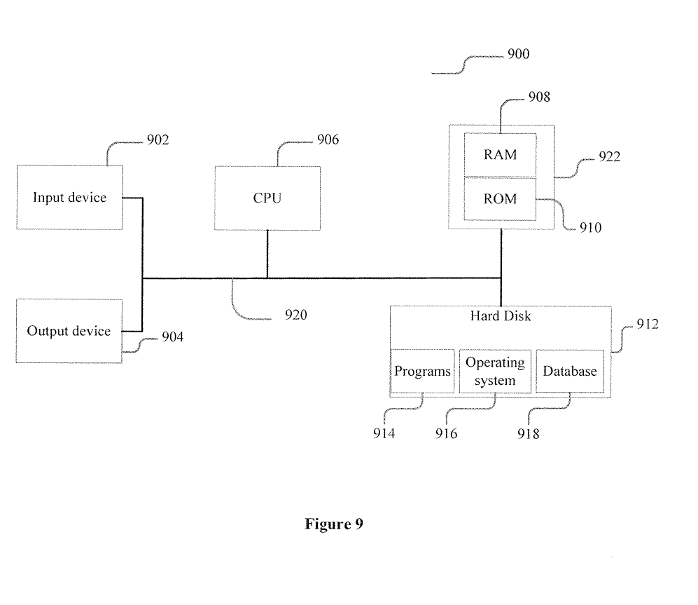

Certain embodiments of a system described in this disclosure are illustrated in FIG. 9. The system 900 comprises a Central Processing Unit (CPU) 906, an input device 902, an output device 904, a memory 922, and a hard disk 912 interconnected by a bus 920. Memory 922 may include a Random Access Memory device (RAM) 908 and a Read-only Memory device (ROM) 910. Hard disk 912 may contain a computer program 914, an operating system 916, and a database 918 stored therein. When executed by the system, the computer program instructs the CPU to perform a method described in this disclosure. It will be understood by a person with ordinary skill in the art that there are many possible configurations of the parts of a computing system, and the illustrative embodiment should not limit the scope of the present disclosure.

The system may be any suitable computing system, including but not limited to personal computers, servers, computing systems comprising a cluster of processors, networked computer, or a personal digital assistant. A computing system may further contain other parts such as a cache memory, a data backup unit, and many other devices.

In certain embodiments, the system is operable to communicate with a database through a network connection to access the information of the reference database and the reference samples therein. In certain embodiments, the system is operable to communicate with a biomarker measurement device, such as a microarray, to access information of biomarker measurement of the test sample.

The computer program of the present disclosure may be executed by being loaded into a system memory and/or a memory storage device through an input device. On the other hand, all or portions of the computer program may also reside in a read-only memory or similar device of memory storage device, such devices not requiring that the computer program first be loaded through input devices. It will be understood by a person with ordinary skill in the art that the computer program or portions of it may be loaded by a processor in a known manner into a system memory or a cache memory or both, as advantageous for execution and used to perform a random sampling simulation.

In certain embodiments of the present disclosure, computer software programs may be stored in a computer server that connects to an end user terminal, an input device or an output device through a data cable, a wireless connection, or a network system. As commonly known in the art, network systems comprise hardware and software to electronically communicate among computers or devices. Examples of network systems may include arrangement over any medium including Internet, Ethernet 10/1000, IEEE 802.11x, IEEE 1394, xDSL, Bluetooth, LAN, WLAN, GSP, CDMA, 3G, PACS, or any other ANSI approved standard.

A person with ordinary skill in the art will recognize that it is common within the art to describe devices and/or processes in the fashion set forth herein, and thereafter use engineering practices to integrate such described devices and/or processes into data processing systems. That is, at least a portion of the devices and/or processes described herein can be integrated into a data processing system via a reasonable amount of experimentation. A person with ordinary skill in the art will recognize that a typical data processing system generally includes one or more of a system unit housing, a video display device, a memory such as volatile and non-volatile memory, processors such as microprocessors and digital signal processors, computational entities such as operating systems, drivers, graphical user interfaces, and applications programs, one or more interaction devices, such as a touch pad or screen, and/or control systems including feedback loops and control motors (e.g., feedback for sensing position and/or velocity; control motors for moving and/or adjusting components and/or quantities). A typical data processing system may be implemented utilizing any suitable commercially available components, such as those typically found in data computing/communication and/or network computing/communication systems.

The herein described subject matter sometimes illustrates different components contained within, or connected with, different other components. It is to be understood that such depicted architectures are merely illustrative, and that in fact many other architectures can be implemented which achieve the same functionality.

Uses of the Methods

This disclosure provides methods, computer programs, computer storage media, and computer systems useful for evaluating and predicting the biological functions of test agents/conditions of interest. The biological functions and effects of the test agents/conditions may be assessed and predicted based on the known effects of the reference agents/conditions. The results of the assessment may be used to direct further research and study of the test agents/conditions. The methods, computer programs, computer storage media, and computer systems described herein can be used to identify agents that affect the same or similar biological functions, or identify agents that cause the same or similar conditions. Information regarding the biological functions and effects of test agents/conditions would be useful for predicting the potential treatment effects of test agents/conditions and identifying therapeutic agents for the prevention and treatment of diseases and disorders.

### EXAMPLES

The following Examples are set forth to aid in the understanding of the present disclosure, and should not be construed to limit in any way the scope of the invention as defined in the claims which follow thereafter.

### Example 1

### Searching for Molecules Having Similar Biological Effects

This example shows the search in a reference database for compounds having similar biological effects as a compound of interest.

Microarray data from a study that investigates the effects of valproic acid on acute myeloblastic leukemia cells OCI/AML2 is assessed using an embodiment of the method described in the present disclosure. Two microarray data is used, in which one microarray analyzes OCI/AML2 cells treated with valproic acid and the other analyzes OCI/AML2 cells treated with a control. Each probe on the microarray detects a gene in the cells, and such gene is considered a tested gene.

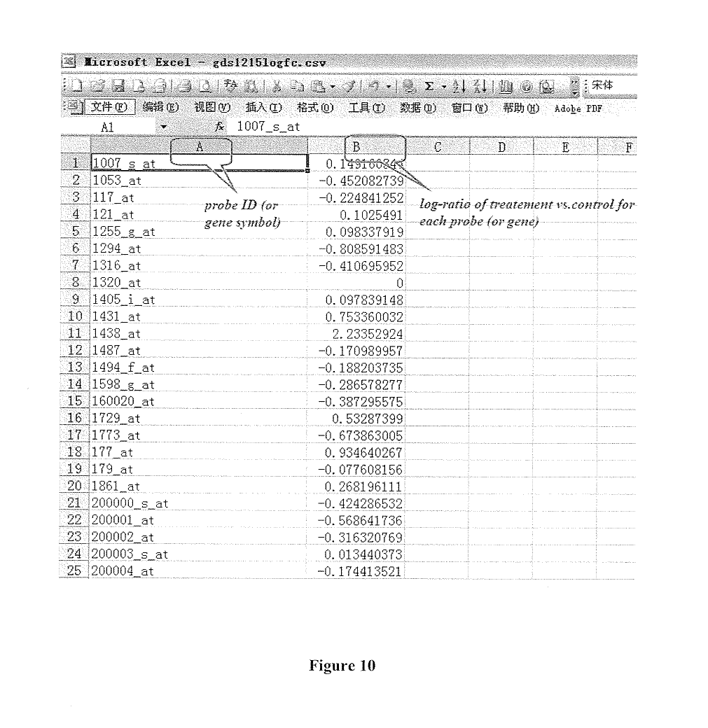

For each probe, ratio of the expression amount detected in valproic acid-treated test sample to the expression amount detected in the control sample is obtained and referred to as R. Arithmetic mean of ratios R of multiple probes is calculated where multiple probes on the microarray detect for the same tested gene. For each tested gene, ratio R is log₂ transformed, and tested genes with a log₂R above 1 or below −1 are selected as target genes. A sample computer interface for tested genes and corresponding log₂ transformed ratios is shown in FIG. 10.

All tested genes are classified into functional domains using biological process category of gene ontology, and all target genes are classified using the same classification. Some functional domains and description of their biological features and functions are shown in FIG. 6.

The statistical significance of enrichment of target genes in each functional domain is calculated using the following Equations:

$$\begin{matrix}f ( k , N , m , n ) = \frac{( \begin{matrix}m \\ k\end{matrix} ) ( \begin{matrix}N - m \\ n - k\end{matrix} )}{( \begin{matrix}N \\ n\end{matrix} )} ; & ( Equation 1 ) \\ P ( k ) = P ( x \geq k ) = 1 - {\sum }_{x = 0}^{k - 1} f ( x , N , m , n ) ; & ( Equation 2 )\end{matrix}$$

wherein f(k, N, m, n) is the probability of finding a total of k target genes in a target functional domain Mᵢ, and P(k) is the p-value for the probability; N represents the total number of tested genes in the microarray, m represents the number of tested genes in the test functional domain tMᵢ, n represents the total number of target genes in the microarray, k represents the number of target genes in target functional domain Mᵢ. Target functional domains having a p-value less than 0.01 are selected as enriched target functional domains, as shown in FIG. 6. The statistical significance may also be set at p-value<0.05.

The target genes of an enriched target functional domain are compared with those genes in microarray data included in the Connectivity Map to determine relevance between test sample data and data in the Connectivity Map. Each enriched target functional domain is further divided into up-regulated group comprising target genes up-regulated in response to treatment of valproic acid, and down-regulated group comprising target genes down-regulated by valproic acid. The relevance is assessed by performing the Kolmogorov-Smirnov test and calculating the KSᵤₚ and KS_{down} scores for each group in an enriched target functional domain Mᵢ using the following equations:

$$\begin{matrix}a = {Max}_{j = 1}^{t} [ \frac{W ( j )}{t} - \frac{V ( j )}{N} ] ; & ( Equation 3 ) \\ b = {Max}_{j = 1}^{t} [ \frac{V ( j )}{N} - \frac{[ W ( j ) - 1 ]}{t} ] ; & ( Equation 4 ) \\ K {S}_{up / down} score = \{ \begin{matrix}a , & ( a > b ) \\ - b , & ( b > a ) ;\end{matrix} & ( Equation 5 )\end{matrix}$$

wherein t is the number of target genes in the up-regulated (or down-regulated group) of the enriched target functional domain Mᵢ, j is the j^{th} target gene of the up-regulated (or down-regulated group) of Mᵢ, W(j) is the rank of gene j among all target genes of the up-regulated or down-regulated group of Mᵢ based on the logarithm log₂R, wherein R is the ratio of the expression amount detected in valproic acid-treated test sample to the expression amount detected in the control sample, V(j) is the rank of gene j among all genes in a microarray in Connectivity Map based on its gene expression profile in response to the reference chemical, N is the total number of tested genes in the microarray in the Connectivity Map. An S-score is calculated using the following equation for each pair of an enriched target functional domain and a reference microarray:

$$S \text{-} score = \{ \begin{matrix}{KS}_{up} - {KS}_{down} , & ( {KS}_{up} \times {KS}_{down} < 0 ) \\ 0 , & ( {KS}_{up} \times {KS}_{down} \geq 0 ) .\end{matrix} \qquad ( Equation 6 )$$

The permutation method is performed to determine the statistical significance of none-zero S-scores. For each calculation of S-score, 1000 hypothetical S-scores are calculated using the above Equations 3-6 in which V(j) is the rank of gene j in the randomly permutated genes in a microarray in the Connectivity Map. The p-value is calculated as the percentage of times when hypothetical S-scores have higher absolute value than real S-scores. S-scores having a p-value less than 0.05 are determined as having statistical significance. For each reference sample in the Connectivity Map, the number of S-scores having statistical significance is counted, and the reference samples are ranked in descending order of such counts. The results are shown in Table 1, and a sample computer interface of the results is shown in FIG. 6. Reference samples (listed by the names of the reference agents) ranked higher in the list are considered having stronger correlation in function with valproic acid than references ranked lower in the list.

TABLE 1

| cMap ID | molecule | dose | | cell line | GO counts |
|---|---|---|---|---|---|
| 1072 | trichostatin A | 1 | uM | MCF7 | 21(20+, 1−) |
| 410 | valproic acid [INN] | 10 | mM | HL60 | 20(20+, 0−) |
| 1000 | vorinostat | 10 | uM | MCF7 | 20(20+, 0−) |
| 1050 | trichostatin A | 100 | nM | MCF7 | 20(20+, 0−) |
| 909 | HC toxin | 100 | nM | MCF7 | 19(19+, 0−) |
| 989 | valproic acid [INN] | 1 | mM | MCF7 | 19(19+, 0−) |
| 332 | trichostatin A | 100 | nM | MCF7 | 18(18+, 0−) |
| 1112 | trichostatin A | 100 | nM | MCF7 | 17(17+, 0−) |
| 866 | ikarugamycin | 2 | uM | MCF7 | 17(17+, 0−) |
| 409 | valproic acid [INN] | 1 | mM | HL60 | 16(16+, 0−) |

Note:
“cMap ID” is the identifier of a microarray in Connectivity Map;
“Molecule” is the reference agent;
“GO counts” means the total counts of enriched target functional domains for each reference sample,
“+” indicates positive S-scores and “−” indicates negative S-scores, and the number before “+” or “−” denotes the number of counts of positive S-scores and negative S-scores, respectively.

Among the reference agents listed in table 1, valproic acid itself appears three times, confirming that the method is capable of identifying relevant compounds with similar biological effects. For the rest, trichostatin A, Vorinostat and HC toxin, though structurally distant are all histone deacetylase inhibitors, which have similar function as valproic acid. Data in the last column of Table 1 show that these reference treatments are almost fully-positively correlated with the query, which is consistent with the fact that they perform a similar function.

### Example 2

### Searching for Molecules that Mimic the Cellular Response to Hypoxia

In this example, microarray data from a study that investigates the effects of hypoxia on gene expression in the MCF-7 cell line is assessed using a method described in the present disclosure. Data from six microarrays is used of which three microarrays have MCF-7 cells affected by hypoxia, the other three have MCF-7 cells without hypoxia, i.e. normoxia. The Connectivity Map is used as the reference database. For each probe, the ratio of the expression amount detected in a hypoxia-affected test sample to the expression amount detected in its normoxia control is obtained and referred to as R. Arithmetic mean of ratio R of multiple probes is calculated where multiple probes on the microarray detect for the same tested gene. Arithmetic mean of the ratio R of the three hypoxia-affected test samples is further calculated for each tested gene. The data is further processed and analyzed using the same method as shown in Example 1. Table 2 shows the top 10 rankings of the reference agents having functional similarity with the state of hypoxia.

TABLE 2

| cMap ID | molecule | dose | | cell line | GO counts |
|---|---|---|---|---|---|
| 573 | deferoxamine [INN] | 100 | uM | MCF7 | 57(57+, 0−) |
| 904 | 5109870 | 25 | uM | MCF7 | 57(57+, 0−) |
| 584 | dimethyloxalylglycine | 1 | mM | PC3 | 52(52+, 0−) |
| 1010 | thioridazine [INN] | 10 | uM | MCF7 | 49(49+, 0−) |
| 460 | deferoxamine [INN] | 100 | uM | PC3 | 48(48+, 0−) |
| 1053 | prochlorperazine | 10 | uM | MCF7 | 46(46+, 0−) |
| | [INN] | | | | |
| 485 | deferoxamine [INN] | 100 | uM | MCF7 | 42(42+, 0−) |
| 977 | wortmannin | 1 | uM | MCF7 | 42(42+, 0−) |
| 1001 | sirolimus [INN] | 100 | nM | MCF7 | 40(40+, 0−) |
| 913 | colforsin [INN] | 50 | uM | MCF7 | 39(39+, 0−) |

All top ten reference agents show fully-positive con-elation with the query, and most of them have been previously reported to have a close relationship with hypoxia. In the results shown in Table 2, deferoxamine appears for three times. Deferoxamine is often used as a hypoxia mimicking agent that simulates the hypoxic state in cells by altering the iron status of hydroxylases. Dimethyloxalylglycine, a non-specific inhibitor of 2-OG-dependent dioxygenase, is another hypoxia mimicking agent. Prochlorperazine has also been reported to have the effects of augmenting hypoxic responsiveness in humans. Colforsin has the ability to mimic the effects of hypoxia with regard to the hypoxia-induced increase in LDH activity. This example demonstrates that the method is suitable for finding, chemicals that cause or mimic a certain biological state.

### Example 3

### Searching for Molecules that Reverse the Expression Pattern of Breast Cancer Cells

In this example, microarray data from a study that investigates expression changes in breast cancer cells having high tumorigenicity is assessed using a method described in the present disclosure. Nine microarray assays are performed, among which six assays analyze tumorigenic cells, the other three analyze non-tumorigenic cells, i.e. normal cells. The Connectivity Map is used as the reference database. For each probe, ratio of the expression amount detected in tumorigenic cells to the expression amount detected in the normal cells is obtained and referred to as R. Arithmetic mean of ratio R of multiple probes is calculated where multiple probes on the microarray are used for detecting, the same tested gene. Arithmetic mean of ratio R of the duplicate assays is further calculated for each gene. The data is further analyzed using the same method as shown in Example 1. Table 3 shows the top 10 ranking of the reference agents having functional antagonism with the state of breast cancer.

TABLE 3

| cMap ID | molecule | dose | | cell line | GO counts |
|---|---|---|---|---|---|
| 448 | trichostatin A | 100 | nM | PC3 | 27(6+, 21−) |
| 1015 | genistein | 10 | uM | MCF7 | 26(0+, 26−) |
| 841 | resveratrol | 10 | uM | MCF7 | 25(0+, 25−) |
| 486 | calmidazolium | 5 | uM | MCF7 | 24(0+, 24−) |
| 164 | dexverapamil [INN] | 10 | uM | MCF7 | 23(0+, 23−) |
| 2 | metformin [INN] | 10 | uM | MCF7 | 23(0+, 23−) |
| 965 | felodipine [INN] | 10 | uM | MCF7 | 20(0+, 20−) |
| 435 | novobiocin [INN] | 100 | uM | PC3 | 20(0+, 20−) |
| 381 | 17-allylamino- | 1 | uM | MCF7 | 20(19+, 1−) |
| | geldanamycin | | | | |
| 383 | cobalt chloride | 100 | uM | MCF7 | 20(0+, 20−) |

Among the top 10 ranked reference agents, 9 are negatively correlated with expression pattern of breast cancer cells. Consistent with the search results, most of the top ranked chemicals are reported to have anti-tumor activities. Trichostatin A is histone deacetylase inhibitor, which has long been investigated as a potential anti-tumor agent against breast cancer. For the rest, genistein, resveratrol, metformin and novobiocin are also reported to have general anti-tumor effects.

### Example 4

### Application of the Method when No Functional domains are Used

The gene expression profile of a test sample is analyzed using the method that does not classify the genes of a test sample into functional domains.

Raw data obtained from a study that investigated the effects of valproic acid on acute myeloblastic leukemia cells OCI/AML2, as illustrated in Example 1, is used in this example. Two microarray data is used, in which one microarray analyzes OCI/AML2 cells treated with valproic acid and the other analyzes OCI/AML2 cells treated with a control. Each probe on the microarray detects a gene in the cells, and such gene is considered a tested gene.

For each probe, ratio of the expression amount of a tested gene of valproic acid-treated test sample to the expression amount of such tested gene of the control sample is obtained and referred to as R. Arithmetic mean of ratios R of multiple probes is calculated where multiple probes on the microarray detect for the same tested gene.

The tested genes are divided into the up-regulated group and the down-regulated group. The tested genes in the up-regulated group are ranked in descending order by the values of the ratios R. The top 10 ranked, top 20 ranked and top 30 ranked genes in the descending ranking are selected as the first, second and third set of target genes in the up-regulated target functional domain, respectively. The tested genes in the down-regulated group are ranked in ascending order by the values of the ratios R. The top 10 ranked, top 20 ranked and top 30 ranked genes in the ascending ranking are selected as the first, second and third set of target genes in the down-regulated target functional domain, respectively.

The three sets of target genes of the up-regulated target functional domains and the three sets of target genes of the down-regulated target functional domains are compared respectively with those genes in microarray data included in the Connectivity Map to determine relevance between test sample data and data in the Connectivity Map. The relevance is assessed by performing the Kolmogorov-Smirnov test and calculating the KSⁱᵤₚ and KSⁱ_{down} scores for each set of target genes in the up-regulated and the down-regulated target functional domains using Equations 3-5 described in Example 1. For a reference sample i, an Si score is calculated using Equation 6 in Example 1. The permutation method is performed to determine the statistical significance of none-zero Si scores wherein for each Si score, 10,000 hypothetical Si scores are calculated as described before. The p-value is calculated as the percentage of times when hypothetical Si scores has higher absolute value than the real Si scores. Si scores having a p-value less than 0.0001 are determined as having statistical significance.

The Si scores having statistical significance are ranked based on their numerical values to identify the max(Si) having the highest numerical value and the min(Si) having the lowest numerical value. For a reference sample i, a relative Si score is calculated by dividing a positive Si score with max(Si) or by dividing a negative Si score with (−min(Si).

The reference samples are ranked in descending order of numerical values of their relative Si scores and the ranking results are shown in Table 4. Reference samples (listed by the names of the reference compounds) ranked higher in the list are considered having stronger correlation in function with valproic acid than references ranked lower in the list.

TABLE 4

| Rank of | Reference Samples | | |
|---|---|---|---|
| reference | Top 10 as | Top 20 as | Top 30 as |
| samples | target genes | target genes | target genes |
| 1 | 17-allylamino- | Butein | quinpirole |
| | geldanamycin | | |
| 2 | NU-1025 | Quinpirole | genistein |
| 3 | monastrol | 17-allylamino- | valproic acid |
| | | geldanamycin | |
| 4 | clofibrate | valproic acid | genistein |
| 5 | thalidomide | N-phenylanthranilic acid | estradiol |
| 6 | geldanamycin | Genistein | 5666823 |
| 7 | 5182598 | Imatinib | trichostatin A |
| 8 | dopamine | trichostatin A | staurosporine |
| 9 | butein | Fluphenazine | rofecoxib |
| 10 | fluphenazine | trichostatin A | wortmannin |

The results show significant changes in the rankings of the reference samples when different numbers of target genes are selected for the analysis. Also, the analysis performs poorly in finding the relevant results. When the first set of target genes are selected, the analysis fails to find any valproic acid treated sample as a relevant reference sample. When the second and third sets of target genes are selected, the analysis is able to find only one valproic acid treated sample as a relevant reference sample. In contrast, the analysis in Example 1 is able to find three valproic acid treated samples as relevant reference samples. These results indicate that when the genes are not classified into functional domains, the method is not effective at finding relevant reference samples.

### Equivalents

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g. “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

We claim:

1. A method for assessing biological effects of a test agent or a test condition, the method comprising:
identifying one or more target biomarkers from a test sample contacted with the test agent or in the test condition;
grouping the one or more target biomarkers into one or more target functional domains according to pre-determined criteria;
identifying one or more reference samples having relevance to the test sample; and
assessing the biological effects of the test agent or the test condition based on the biological effects of one or more reference agents or reference conditions on the one or more reference samples.

2. The method of claim 1, wherein identifying one or more target biomarkers comprises:
receiving a set of test data representing one or more characteristics of one or more biomarkers of the test sample contacted with the test agent or in the test condition, and a set of control data representing one or more characteristics of one or more biomarkers of the test sample not contacted with the test agent or in the test condition;
calculating changes between the test data and the control data for the one or more biomarkers; and
selecting one or more target biomarkers of the test sample, wherein each target biomarker shows changes between the test data and the control data.

3. The method of claim 2, wherein each target biomarker shows statistically significant changes between the test data and the control data.

4. The method of claim 2, wherein the changes in the test data and the control data for the one or more biomarkers are calculated as log₂R, wherein R is the ratio of the test data to the control data for the one or more biomarkers.

5. The method of claim 1, wherein the pre-determined criteria is based on biological features and functions of the target biomarkers.

6. The method of claim 5, wherein the biological features and functions include molecular or cellular functions, metabolic pathways, biological processes, cellular localizations, or physiological functions.

7. The method of claim 1, wherein grouping the one or more target biomarkers into one or more target functional domains further comprises identifying one or more enriched target functional domains.

8. The method of claim 7, wherein identifying one or more enriched target functional domains comprises:
calculating the probability of appearance of the one or more target biomarkers in the target functional domain;
calculating the statistical significance of said probability of appearance;
repeating the above calculation of the probability of appearance and the statistical significance for each target functional domain of the test sample; and
selecting one or more enriched target functional domains.

9. The method of claim 8, wherein the probability of appearance and the statistical significance of the one or more target biomarkers are determined according) to the following equations:
$$\begin{matrix}f ( k , N , m , n ) = \frac{( \begin{matrix}m \\ k\end{matrix} ) ( \begin{matrix}N - m \\ n - k\end{matrix} )}{( \begin{matrix}N \\ n\end{matrix} )} ; \\ P ( k ) = P ( x \geq k ) = 1 - {\sum }_{x = 0}^{k - 1} f ( x , N , m , n ) ;\end{matrix}$$
wherein
f(k, N, m, m) is the probability of appearance of a total of k target biomarkers in a target functional domain Mᵢ, and P(k) is the p-value representing the statistical significance for the target functional domain Mᵢ;
k is the number of target biomarkers in the target functional domain Mᵢ;
N is the total number of biomarkers in the test sample;
m is the number of biomarkers of the test sample in the test functional domain tMᵢ that corresponds with the target functional domain Mᵢ; and
n is the total number of target biomarkers in the test sample.

10. The method of claim 1, wherein said identifying one or more reference samples comprises:
for the one or more target biomarkers in a target functional domain, calculating the KS score for the one or more target biomarkers with respect to a reference sample according to the following equations:
$$\begin{matrix}a = {Max}_{j = 1}^{t} [ \frac{W ( j )}{t} - \frac{V ( j )}{N} ] ; \\ b = {Max}_{j = 1}^{t} [ \frac{V ( j )}{N} - \frac{[ W ( j ) - 1 ]}{t} ] ; \\ K S score = \{ \begin{matrix}a , & ( a > b ) \\ - b , & ( b > a ) ;\end{matrix}\end{matrix}$$
wherein
t is the number of target biomarkers in the target functional domain Mᵢ;
j is the jth target biomarker in the target functional domain Mᵢ;
W(j) is the rank of target biomarker j among all target biomarkers in the target functional domain Mᵢ based on the change in the characteristics of the target biomarkers;
V(j) is the rank of the reference biomarker j, which is the same biomarker as the target biomarker j, among the reference biomarkers of the reference sample based on the change in the characteristics of the reference biomarkers; and
N is the total number of reference biomarkers in the reference sample.
determining the statistical significance of the above calculated KS score;
repeating the above calculation of KS score and determination of statistical significance for each target functional domains of the test sample with respect to every reference sample; and
selecting the reference samples that have at least one statistically significant KS score.

11. The method of claim 10, wherein the statistical significance of the KS score is represented by the p-value calculated as the percentage of times when the absolute value of a hypothetical KS score is higher than the absolute value of the KS score, and wherein the hypothetical KS score is calculated using the K-S Test based on randomly ranked reference biomarkers of the reference sample.

12. The method of claim 10, wherein identifying one or more reference samples further comprises:
counting the number of target functional domains that have statistically significant KS scores with respect to every reference sample; and
ranking the reference samples based on their numbers of statistically significant KS scores.

13. The method of claim 7, wherein said identifying one or more reference samples comprises:
for the one or more target biomarkers in an enriched target functional domain, calculating the KS score for the one or more target biomarkers with respect to a reference sample according to the following equations:
$$\begin{matrix}a = {Max}_{j = 1}^{t} [ \frac{W ( j )}{t} - \frac{V ( j )}{N} ] ; \\ b = {Max}_{j = 1}^{t} [ \frac{V ( j )}{N} - \frac{[ W ( j ) - 1 ]}{t} ] ; \\ K S score = \{ \begin{matrix}a , & ( a > b ) \\ - b , & ( b > a ) ;\end{matrix}\end{matrix}$$
wherein
t is the number of target biomarkers in the enriched target functional domain;
j is the jth target biomarker in the enriched target functional domain;
W(j) is the rank of target biomarker j among all target biomarkers in the enriched target functional domain based on the change in the characteristics of the target biomarkers:
V(j) is the rank of the reference biomarker j, which is the same biomarker as the target biomarker j, among the reference biomarkers of the reference sample based on the change in the characteristics of the reference biomarkers; and
N is the total number of reference biomarkers in the reference sample.
determining the statistical significance of the above calculated KS score;
repeating the above calculation of KS score and determination of statistical significance for each enriched target functional domains of the test sample with respect to every reference sample; and
selecting the reference samples that have at least one statistically significant KS score.

14. The method of claim 13, wherein identifying one or more reference samples further comprises:
counting the number of enriched target functional domains that have statistically significant KS scores with respect to every reference sample; and
ranking the reference samples based on their numbers of statistically significant KS scores.

15. The method of claim 1, wherein identifying one or more reference samples comprises:
for the one or more target biomarkers in a target functional domain, separating the one or more target biomarkers into an up-regulated group and a down-regulated group;
calculating a KS score for the up-regulated group and a KS score for the down-regulated group with respect to a reference sample according to the following equations:
$$\begin{matrix}a = {Max}_{j = 1}^{t} [ \frac{W ( j )}{t} - \frac{V ( j )}{N} ] ; \\ b = {Max}_{j = 1}^{t} [ \frac{V ( j )}{N} - \frac{[ W ( j ) - 1 ]}{t} ] ; \\ K S score = \{ \begin{matrix}a , & ( a > b ) \\ - b , & ( b > a ) ;\end{matrix}\end{matrix}$$
wherein
t is the number of target biomarkers in the up-regulated group (or down-regulated group);
j is the jth target biomarker in the up-regulated group (or down-regulated group);
W(j) is the rank of target biomarker j among all target biomarkers in up-regulated group (or down-regulated group) based on the change in the characteristics of the target biomarkers;
V(j) is the rank of the reference biomarker j, which is the same biomarker as the target biomarker j, among the reference biomarkers of the reference sample based on the change in the characteristics of the reference biomarkers; and
N is the total number of reference biomarkers in the reference sample;
calculating the S-score for the target functional domain according to the following equation:
$$S \text{-} score = \{ \begin{matrix}{KS}_{up} - {KS}_{down} , & ( {KS}_{up} \times {KS}_{down} < 0 ) \\ 0 , & ( {KS}_{up} \times {KS}_{down} \geq 0 ) ;\end{matrix}$$
wherein KSᵤₚ is the KS score for the up-regulated group, and
KS_{down} is the KS score for the down-regulated group;
calculating the p-value of the S-score of the target functional domain;
repeating the above calculation of S-score and p-value for each target functional domains of the test sample with respect to every reference sample; and
selecting the reference samples that have at least one statistically significant S-score.

16. The method of claim 15, wherein identifying one or more reference samples further comprises:
counting the number of statistically significant S-scores for the test sample with respect to every reference sample; and
ranking the reference samples based on their numbers of statistically significant S-scores.

17. The method of claim 7, wherein identifying one or more reference samples comprises:
for the one or more target biomarkers in an enriched target functional domain, separating the one or more target biomarkers into an up-regulated group and a down-regulated group;
calculating a KS score for the up-regulated group and a KS score for the down-regulated group with respect to a reference sample according to the following equations:
$$\begin{matrix}a = {Max}_{j = 1}^{t} [ \frac{W ( j )}{t} - \frac{V ( j )}{N} ] ; \\ b = {Max}_{j = 1}^{t} [ \frac{V ( j )}{N} - \frac{[ W ( j ) - 1 ]}{t} ] ; \\ K S score = \{ \begin{matrix}a , & ( a > b ) \\ - b , & ( b > a ) ;\end{matrix}\end{matrix}$$
wherein
t is the number of target biomarker-s in the up-regulated group (or down-regulated group);
j is the jth target biomarker in the up-regulated group (or down-regulated group);
W(j) is the rank of target biomarker j among all target biomarkers in up-regulated group (or down-regulated group) based on the change in the characteristics of the target biomarkers;
V(j) is the rank of the reference biomarker j, which is the same biomarker as the target biomarker j, among the reference biomarkers of the reference sample based on the change in the characteristics of the reference biomarkers; and
N is the total number of reference biomarkers in the reference sample;
calculating the S-score for the enriched target functional domain according to the following equation:
$$S \text{-} score = \{ \begin{matrix}{KS}_{up} - {KS}_{down} , & ( {KS}_{up} \times {KS}_{down} < 0 ) \\ 0 , & ( {KS}_{up} \times {KS}_{down} \geq 0 ) ;\end{matrix}$$
wherein KSᵤₚ is the KS score for the up-regulated group, and
KS_{down} is the KS score for the down-regulated group;
calculating the p-value of the S-score of the enriched target functional domain.
repeating the above calculation of S-score and p-value for each enriched target functional domains of the test sample with respect to every reference sample; and
selecting the reference samples that have at least one statistically significant S-score.

18. The method of claim 1, wherein assessing the biological effects comprises:
retrieving the biological effects of the one or more reference agents or reference conditions on the one or more identified reference samples; and
assessing the biological effects of the test agent or the test condition based on the biological effects of the one or more reference agents or reference conditions.

19. A computer readable storage medium having a computer program product encoded thereon, wherein said computer program product when executed by a computer instructs the computer to execute a method for assessing biological effects of a test agent or a test condition, which comprises:
identifying one or more target biomarkers from a test sample contacted with the test agent or in the test condition;
grouping the one or more target biomarkers into one or more target functional domains according to pre-determined criteria;
identifying one or more reference samples having relevance to the test sample;
assessing the biological effects of the test agent or the test condition based on the biological effects of the one or more reference agents or reference conditions on the one or more reference samples; and
outputting the assessing results.

20. A system for assessing biological effects of a test agent or a test condition, comprising:
one or more input devices, one or more output devices, one or more processors, and one or more memory devices storing therein one or more operating systems, one or more computer programs, and one or more optional databases, interconnected by a bus; wherein, the computer programs comprising:
one or more instructions to cause the one or more processors to identify one or more target biomarkers from a test sample contacted with the test agent or in the test condition;
one or more instructions to cause the one or more processors to group the one or more target biomarkers into one or more target functional domains according to predetermined criteria;
one or more instructions to cause the one or more processors to identify one or more reference samples having relevance to the test sample;
one or more instructions to cause the one or more processors to assess the biological effects of the test agent or the test condition based on the biological effects of the one or more reference agents or reference conditions on the one or more reference samples; and
one or more instructions to cause the one or more processors to output the assessing results.
